(19) United States
(12) Patent Application Publication
(10) Pub. No.: US 2014/0342427 A1
(43) Pub. Date: Nov. 20, 2014

(54) PHOTONIC NANOANTENNA MEDIATED GENE CIRCUIT RECONFIGURATION

(71) Applicant: THE REGENTS OF THE UNIVERSITY OF CALIFORNIA, Oakland, CA (US)

(72) Inventors: Luke P. Lee, Orinda, CA (US); Somin Eunice Lee, Fair Oaks, CA (US)

(73) Assignee: THE REGENTS OF THE UNIVERSITY OF CALIFORNIA, Oakland, CA (US)

(21) Appl. No.: 14/288,081

(22) Filed: May 27, 2014

Related U.S. Application Data
(63) Continuation of application No. PCT/US2012/067103, filed on Nov. 29, 2012.
(60) Provisional application No. 61/564,556, filed on Nov. 29, 2011.

(51) Int. Cl. C12N 13/00

(52) U.S. Cl. CPC C12N 13/00
USPC 435/173.1; 428/403

(57) ABSTRACT

A selectively addressable optical biomolecular carrier and its method of use for reconfiguring gene circuits are described. One carrier is a plasmon resonant nanoantenna formed from a gold metal nanorod coated with a cationic phospholipid bilayer with an aspect ratio between 2.0 and 8.0 and plasmon resonance wavelength in the near infrared range. Biomolecules such as siRNA adhere to the carrier and are introduced into a cell. The biomolecules are released from the nanoantenna carriers with exposure to light at the plasmon resonance wavelength. The nanoantenna efficiently converts absorbed optical energy to surface localized heat releasing the biomolecules at a time determined by the user. The carrier can be used to modify gene circuits by allowing temporal control over the genes within a selected gene circuit through the optical release of interfering nucleotides. Optical silencing of endogenous genes with siRNA released from nanoantenna carriers was used to illustrate the methods.

### CROSS-REFERENCE TO RELATED APPLICATIONS

This application is a 35 U.S.C. §111(a) continuation of PCT international application number PCT/US2012/067103 filed on Nov. 29, 2012, incorporated herein by reference in its entirety, which claims priority to, and the benefit of, U.S. provisional patent application Ser. No. 61/564,556 filed on Nov. 29, 2011, incorporated herein by reference in its entirety. Priority is claimed to each of the foregoing applications.

The above-referenced PCT international application was published as PCT International Publication No. WO 2013/082304 on Jun. 6, 2013, incorporated herein by reference in its entirety.

### STATEMENT REGARDING FEDERALLY SPONSORED RESEARCH OR DEVELOPMENT

This invention was made with Government support under Grant Number EY018241 awarded by the National Institutes of Health (NIH). The Government has certain rights in the invention.

### INCORPORATION-BY-REFERENCE OF MATERIAL SUBMITTED ON A COMPACT DISC

Not Applicable

### BACKGROUND OF THE INVENTION

1. Field of the Invention

This invention pertains generally to modulated delivery and artificial gene expression control systems, and more particularly to a system and method for the dynamic perturbation, reconfiguration and reconstruction of gene circuits using optically addressable siRNA-Au nanoantennas as optical inputs to existing circuit connections in living cells.

2. Description of Related Art

The genetic circuitry of an organism includes the connections of its array of genes in its genome to its observed patterns of phenotypic expression. The genotype of an organism is determined by the information that is encoded in its DNA sequence, while the phenotype is determined by the expression of those genes in the genome within the biological context of the organism. The genetic circuitry of an organism interprets the environment inside and outside of the cell and then orchestrates the patterns of expression in response. These patterns reflect the temporal flow of information from DNA to RNA to protein to metabolites.

Transcriptional or genetic regulatory circuits are networks of functional clusters of genes that influence the expression of certain genes through inducible transcription factors and other regulatory elements. Genetic regulatory circuits are similar to electronic circuits in several ways. Genetic circuits can be seen as cellular networks that are “wired” through interactions between “component parts” through proteins, mRNA, and secondary signaling molecules etc. The circuits have an input and output logic that can be switched “on” or “off” or increase or decrease regulated rates of activity over time.

On the input side of a typical genetic circuit, extracellular or intracellular signals are detected through binding with a receptor molecule that initiates a signal to regulatory molecules through transduction and the regulatory molecules ultimately initiate or influence the rate of transcription of genes in the network. The biological context normally determines the functional requirements of gene circuits. Transcription units can also be one or more genes that are transcribed as a unit and expressed in a coordinated manner. Cascades of gene expression produce output signals from the transcription units.

On the output side, transcription can start a cascade of gene expression generating many different types of products. A messenger (mRNA) molecule that has a sequence that is complementary to the transcribed DNA strand is the normal initial output of a transcription unit. The mRNA is then translated to produce an encoded protein product that may be processed further. A wide variety of protein products may be produced including enzyme products and secondary signaling products.

Positive and negative regulators can also exert control over gene expression. Negative regulators terminate expression by blocking expression. Positive gene regulators induce gene expression directly or by the removal of a negative regulator.

Today hundreds of diverse genetic circuits or sub-circuits with different regulatory mechanisms have been characterized experimentally. The principal aim of experimental and theoretical studies of gene circuits is to understand the relationship between system structure and function. For example, patterns of regulation in some simple gene circuits can be understood in terms of the functional requirements for biosynthesis and catabolism.

However, many gene circuits are incomplete or only partially understood. The complexity of the system and the specific properties of the components and their pattern of interactions make it difficult to determine how gene-circuit design relates to gene-circuit function in many circuits. The recognition of system design principles and the analysis of patterns also require modeling systems where all of the components and their interactions are known.

The manipulation of certain elements in gene circuits has been used in the study of transcription and for potential therapeutic applications. The recent recognition of different design principles in transcriptional regulation have benefited from the development of synthetic regulatory circuits. One purpose of synthetic biology is to develop a greater understanding of biological design principles by building synthetic circuits and then studying their behavior in cells. The precise perturbation of gene circuits and the direct observation of signaling pathways in living cells are essential for both fundamental biology and translational medicine.

Increased understanding of information processing and the operation of gene circuits should also advance therapeutic strategies for reconfiguring gene circuits involved in disease progression and cancer. One major challenge is probing native gene circuits with high signal fidelity. Top-down probing approaches utilize an external input signal and essentially treat a living cell as an input-output “black box.” Ideally, the output signal should be directly correlated to the input signal. However, signal distortions such as time delays, noise, and signal magnitude reductions, can frustrate this input-output relationship, hindering temporally precise modulation of gene circuits and limiting dynamic reconfiguration of gene circuits in living cells.

Accordingly, there is a need for a method for reliably producing transcriptional activation or deactivation or rate manipulations in gene circuits in cells to produce engineered gene circuits useful for fundamental bioscience, bioengineering, and medical applications. The present invention satisfies this need as well as others and is generally an improvement over the art.

### BRIEF SUMMARY OF THE INVENTION

The present invention provides for on-demand optical gene circuit reconfiguration that is enabled by resonant optical nanoparticle carriers (referred to as biomolecular nanoantennas) functioning as selectively addressable optical receivers and biomolecular emitters of small interfering RNA (siRNA) or other bioactive molecules within a cell.

By receiving and focusing the energy of freely propagating optical radiation into localized energy, a resonant optical nanoantenna enables control of absorption and emission at the nanometer-scale. Resonant optical nanoantennas may be utilized to efficiently convert absorbed optical energy into surface-localized heat. When biomolecules are functionalized to the surface of the nanoantenna, this optically generated, surface-localized heat can be used to liberate (i.e. emit) biomolecules from the nanoantenna. In this context, an optical nanoantenna functions as an optical receiver and a biomolecular emitter, enabling wireless access to circuit connections of living cells.

Among the various nanoantenna structures available, a rod-shaped nanoantenna of gold metal is preferred because of its large absorption cross-section, narrow spectral bandwidth of the longitudinal plasmon resonance band, and tunable longitudinal plasmon resonance wavelength in the near infrared (NIR) regime, where cells are essentially transparent. The optical properties of rod-shaped gold nanoparticles are dominated by its plasmon resonance. At resonance, the optical near-field shows two regions of high field intensity due to charge accumulation at each end of the nanoantenna. These regions of high field intensity indicate that the nanoantenna functions as an optical receiver to focus freely propagating optical fields down to nanometer-scale dimensions. This antenna effect is prominent at the resonance state of the nanoantenna, where the excitation wavelength is matched with the plasmon resonance wavelength.

The surface of the preferred biomolecular nanoantenna is modified with a cationic lipid layer and preferably a phospholipid bilayer. Negatively charged siRNA is functionalized to the surface of the lipid layer forming the final biomolecular nanoantenna carrier. This lipid layer is preferred because it facilitates introduction of the carriers into cells and the adsorption of biomolecules. The siRNA-Au nanoantennas fulfill dual functions as selectively addressable optical receivers and biomolecular emitters of small interfering RNA (siRNA) or other adsorbed molecules.

The tunable absorption of gold nanoparticles in the infrared spectral region and straightforward surface functionalization has led to applications in intracellular delivery and photo release of short RNAs, enabling bidirectional photothermal modulation of specific genes through RNA interference. The siRNA or other adsorbed biomolecule is emitted from the biomolecular nanoantenna carrier when the antenna effect is “on” at the resonance state, and no siRNA is emitted when the antenna effect is “off” at the non-resonance state. Electric fields were calculated using excitation λ=780 nm or λ=650 nm.

Accordingly, many different carriers can be made from selected nanoparticles with different non-overlapping plasmon resonance wavelengths. Each group of nanoantenna particles can be coated with a lipid bilayer and specific biomolecules can be adsorbed to the nanoantenna carrier. Each type of biomolecule can be released with the exposure to light of a specific plasmon resonance wavelength at a time controlled by the user.

Dynamic optical circuit reconfiguration is enabled by resonant biomolecular nanoantennas as optical inputs to existing circuit connections of living cells, forming photonic gene circuits. Resonant biomolecular nanoantennas function as selectively addressable optical receivers and emitters of specific biologically active molecules that can have specific influences on selected gene circuits. For example, transcription factor proteins, signaling proteins, and sequence specific RNA and DNA molecules can be introduced and temporally activated to initiate, terminate or bypass cellular functions. Transcription of genes in a circuit can be activated or deactivated out of sequence or can remain active or inactive by interfering with the natural circuitry. The precise control of the perturbation and reconfiguration of gene circuits in living cells by optically addressable siRNA-Au nanoantennas is illustrated.

It was shown that photonic gene circuits are modular, enabling sub-circuits to be combined to form large-scale circuit configurations. Photonic gene circuits open new avenues for engineering functional gene circuits useful for fundamental bioscience, bioengineering, and medical applications. As a future therapeutic strategy, photonic gene circuits could be used to probe, identify, and reconfigure malfunctioning gene circuits involved in disease progression and cancer. Intracellular approaches that enable temporally precise modulation of native gene circuits and dynamic reconfiguration of existing circuit connections will have widespread applications in fundamental bioscience, bioengineering, and medicine.

Accordingly, an aspect of the invention is to provide a biomolecular carrier that is precise, predictable, and easy to introduce into a cell.

Another aspect of the invention is to provide a biomolecular carrier that provides temporal control over the release of biomolecules that will signal or modulate selected cellular transcription or translation activities in a cell.

Another aspect of the invention is to provide a method for on-switch” or “off-switch” or “rate control” and similar control over genes and gene circuits.

A further aspect of the invention is to provide a method for engineering photonic gene circuits or for reconfiguring functioning and malfunctioning gene circuits.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWINGS

The invention will be more fully understood by reference to the following drawings which are for illustrative purposes only:

FIG. 1 is a flow diagram of a method of forming biomolecular emitters with selected siRNA and the precise control of the perturbation and reconfiguration of gene circuits in living cells by optically addressable siRNA-Au emitters according to one embodiment of the invention.

FIG. 2 through FIG. 2E illustrate simple “off switch,” “on-switch” and “pulse switch” photonic gene circuits according to the invention.

FIG. 3A and FIG. 3B illustrate a p65 OFF-switch photonic gene circuit in single HeLa cells according to the invention, including circuit diagram, logic table and flow cytometry data.

FIG. 4A through FIG. 4C illustrate an ON-switch photonic gene circuit according to the invention constructed using a modular OFF-switch sub-circuit. IκB and p65 were chosen to represent Y and X, respectively.

FIG. 5A through FIG. 5D illustrate a PULSE-switch photonic gene circuit according to the invention constructed using multiple, independently operating sub-circuits.

FIG. 6 is a graph of UV-VIS spectra of gold rod-shaped biomolecular nanoantennas with longitudinal plasmon resonances at 780 nm and 650 nm.

FIG. 7A through FIG. 7C show the characterization of thermally liberated siRNA with a UV-VIS spectra of siRNA-nanoantennas as the temperature increased from 20° to 70° C. and UV-VIS spectra of control nanoantennas as temperature increased from 20° to 70° C. The longitudinal plasmon resonance wavelength of control nanoantennas and siRNA-nanoantennas as a function of temperature is also shown.

FIG. 8A and FIG. 8B depict the characterization of optically liberated siRNA with matching FAM-siRNA-nanoantennas with longitudinal plasmon resonance 780 nm addressed using 785 nm wavelength light and matching FAM-siRNA-nanoantennas with longitudinal plasmon resonance 650 nm addressed using 660 nm wavelength light, and mismatch controls.

FIG. 9A through FIG. 9C diagrammatically illustrate a method of optical gene silencing according to one embodiment of the invention.

### DETAILED DESCRIPTION OF THE INVENTION

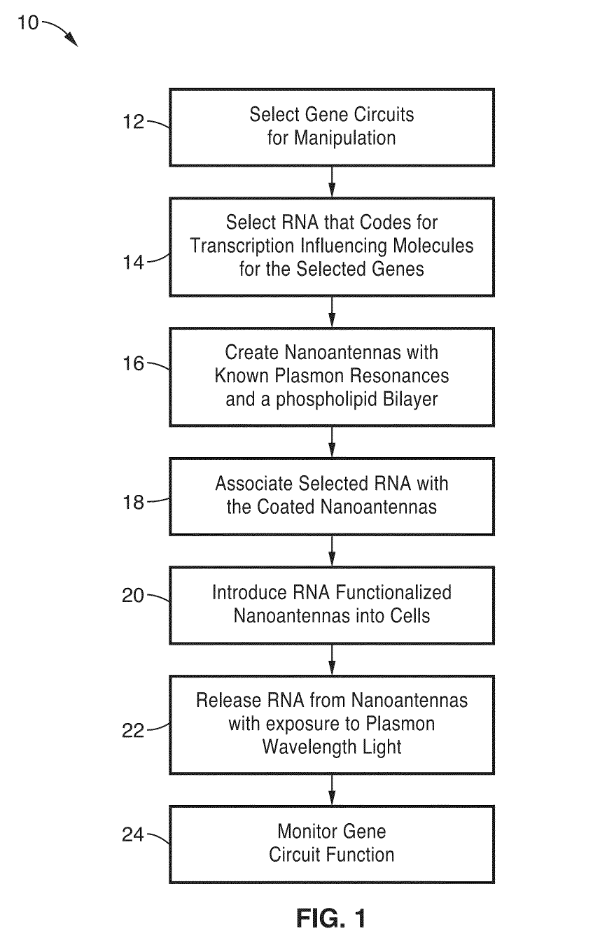

Referring more specifically to the drawings, for illustrative purposes one embodiment of the present invention is depicted in the carrier and methods generally shown in FIG. 1 through FIG. 9C. It will be appreciated that the methods may vary as to the specific steps and sequence and the apparatus may vary as to structural details, without departing from the basic concepts as disclosed herein. The steps depicted and/or used in methods herein may be performed in a different order than as depicted in the figures or stated. The steps are merely exemplary of the order these steps may occur. The steps may occur in any order that is desired, such that it still performs the goals of the claimed invention.

The present invention provides methods for exerting specific temporal control over selected gene circuits with the use of phospholipid bilayer coated nanoplasmonic gold nanorods and electrostatically coupled biomolecules like RNA. The lipid functionalized nanoplasmonic gold nanorods are preferably sized so that they can be activated by exposure to light in the near infrared (NIR) range because human tissues and cells are virtually transparent at 800 nm to 1300 nm wavelengths. The selected nucleic acids can be adsorbed on to the phospholipid bilayer coating of the nanorod carrier and introduced into a cell. The electrostatic interaction of the cargo molecules with the nanorod bilayer is destabilized upon exposure of light at the plasmon resonance wavelength because the optical energy is absorbed and converted to thermal energy. The emitted cargo biomolecules are then available to influence cellular activity in a predictable way.

Turning now to FIG. 1, one method 10 for influencing selected gene circuits is schematically shown. At block 12, a gene circuit for analysis and manipulation is identified. The gene circuit that is selected can be simple or complex. The nature of the manipulation of the selected gene circuit can take various forms such as transcription initiation, transcription repression or rate reduction or acceleration or signal production. The gene circuit manipulations can also be temporal. For example, genes can be activated or deactivated out if sequence or independently. The gene circuits can also be composed of groups of multiple coordinated genes.

At block 14 of FIG. 1 a biomolecule is selected that will produce the intended influence on the genetic circuit that was selected at block 12. Biomolecule cargo can be various types of RNA, DNA, oligonucleotides or proteins that influence transcription either directly or through secondary influences, for example. Small interfering RNA (siRNA) is used as an illustration. The RNA can be translated to produce a protein that promotes or represses transcription of a particular gene or cascade of genes or signals other cellular events. Exogenous foreign genes could also be delivered. It will be seen that the selection of gene circuit and desired influence on the circuit will determine the type molecule that is selected as well as the sequence of a selected nucleic acid. Transcription factor proteins and signaling proteins can also be selected.

While a single type of biomolecule is selected in the embodiment shown in FIG. 1, more than one type of biomolecule can be selected for introduction. For example, transcription factor proteins and siRNA's can be selected for simultaneous control of associated processes or unrelated processes.

Once the gene circuits and the effector biomolecule are selected at blocks 12 and 14, the plasmonic nanoantenna emitter is created at block 16. Preferably, gold metal nanorods with aspect ratios of between approximately 2.0 to approximately 8.0 are provided. The nanorods within this range should have characteristic plasmon resonance wavelengths within the NIR range.

The surface of the nanorod (nanoantenna) is preferably functionalized with a cationic lipid layer, preferably a phospholipid bilayer. A cationic phospholipid bilayer is preferred because it shows no cytotoxicity under physiological conditions and the plasmonic properties of the nanorod or nanoparticle are maintained. In addition, the phospholipid bilayer assists in the introduction of the nanoantenna carriers into the cells of interest. While a phospholipid bilayer is preferred, other coatings can be used. In addition, some biomolecules that can be selected will directly adsorb to the nanorod antennas making the nanorod lipid coating unnecessary.

At block 18 of FIG. 1, the selected biomolecule at block 14 is associated with the coated nanorod antenna carrier. The bilayer that coats the gold nanorod has a positively charged surface that can be used to adsorb negatively charged biomolecules such as RNA and DNA oligonucleotides, siRNA, transcription factors, proteins and various drugs. The molecules can either bind to the phospholipid surface or be incorporated into the bilayer.

The assembled functionalized nanoantennas produced at block 18 are introduced into cells with the gene circuits of interest at block 20. Groups of nanoantennas based on nanorods with different resonance wavelengths that each have different adsorbed biomolecules can be introduced into the cell simultaneously or sequentially.

Release of the biomolecules from the nanoantennas at block 22 is produced by the exposure of the cells to plasmon wavelength light for the particular nanorod. The energy from the light is converted by the nanorod to heat and the electrostatic interactions of each biomolecule with the bilayer are destabilized and the biomolecules are released. The timing of the release is determined by the user through the control of the timing of the light exposure.

The function of the gene circuit is monitored to observe the effect of the delivered biomolecules at block 24.

Although the formation and use of a single type of nanoantenna biomolecular carrier is illustrated in FIG. 1, it will be understood that more than one type of carrier with each type having a different plasmon resonant wavelength can be used and coordinated with different types of biomolecules. Each type of biomolecule can be released from a specific carrier with the exposure of a particular wavelength of light.

In one embodiment, a single type of carrier has several different types of biomolecules adhered to the surface that are released at the same time with a single plasmon wavelength light exposure. For example, this could include siRNA's directed to several different genes in a selected gene circuit that are released at the same time to influence the genes of the circuit simultaneously.

In another embodiment, a first group of coated plasmon resonant nanoparticles with one type of biomolecule adsorbed to the coating and a second group of a second type of coated plasmon resonant nanoparticle with a second type of adsorbed biomolecule are prepared and introduced into cells. The two carriers can be introduced into the cells at the same time or sequentially. The first biomolecules are released with exposure to one wavelength of light while the second biomolecules in the second carrier are inactive. The second group of biomolecules is then released with the exposure to a second wavelength of light at the plasmon resonance wavelength of the second nanoantenna carrier.

Likewise, three or more types of nanoantenna carriers with discrete plasmon wavelengths can be used to introduce three or more different types of biomolecules into a cell. Each biomolecule can be released from the carrier at selected times and cellular locations with the exposure of the cells to the corresponding plasmon wavelength light.

Optical input signals are a promising interface to gene circuit connections. To date, current optical methods to interface living cells have so far relied on genomic modifications (i.e. mutations, overexpression) to permanently encode living cells with light-responsive genes, thus limiting dynamic circuit reconfiguration. Here, on-demand optical circuit reconfiguration is enabled by resonant biomolecular nanoantennas as optical inputs to existing circuit connections, forming photonic gene circuits. Photonic gene circuits are modular, allowing sub-circuits to be combined to form large-scale circuit configurations on-demand. The ability to optically apply input signals and reconfigure existing gene circuit connections should be transformative for engineering functional gene circuits in complex, naturally occurring living systems.

The invention may be better understood with reference to the accompanying examples, which are intended for purposes of illustration only and should not be construed as in any sense limiting the scope of the present invention as defined in the claims appended hereto.

### Example 1

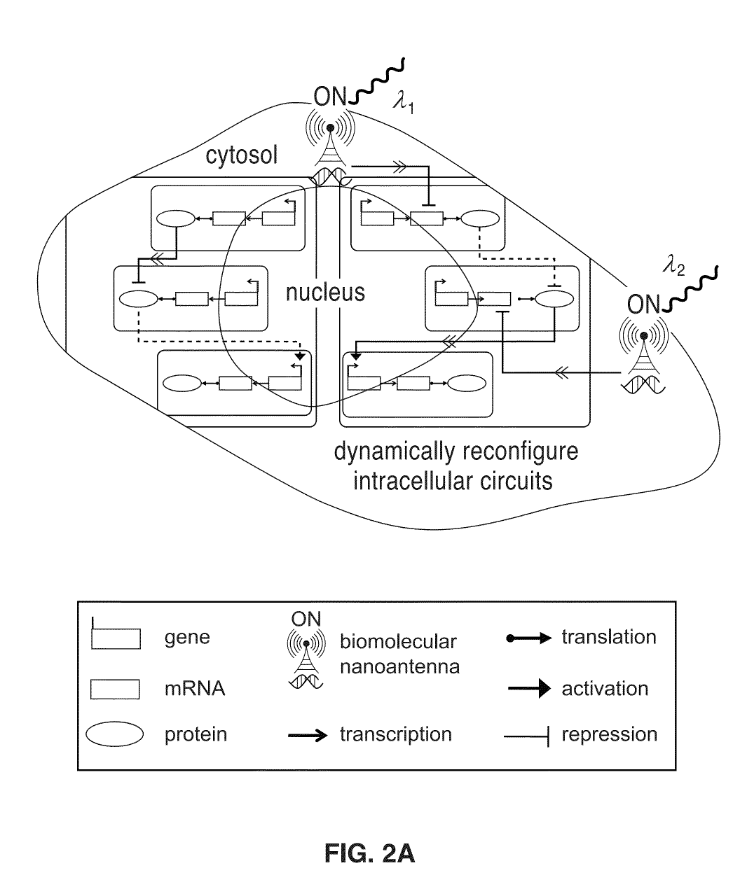

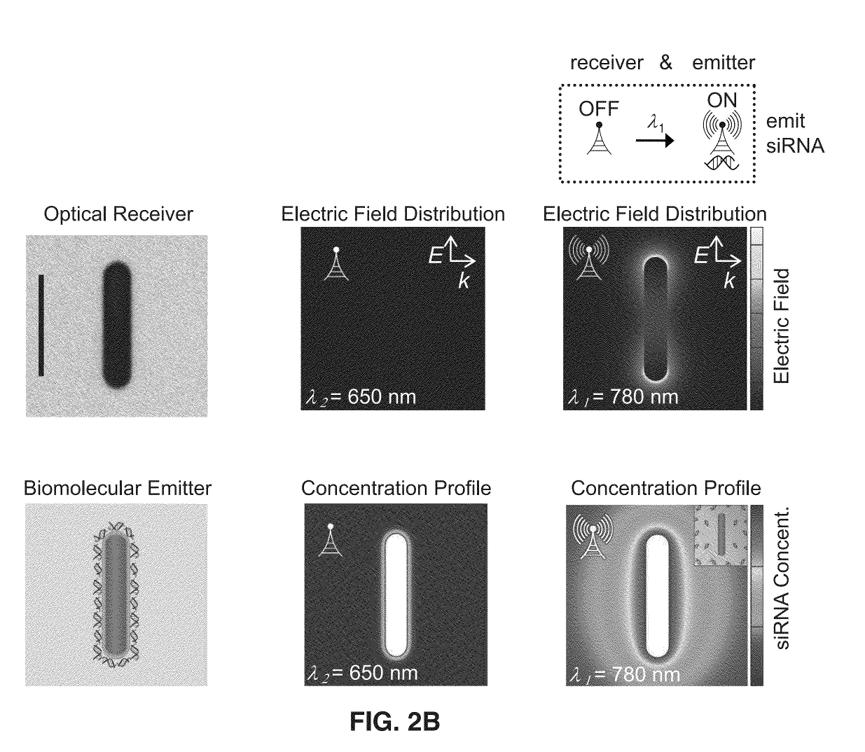

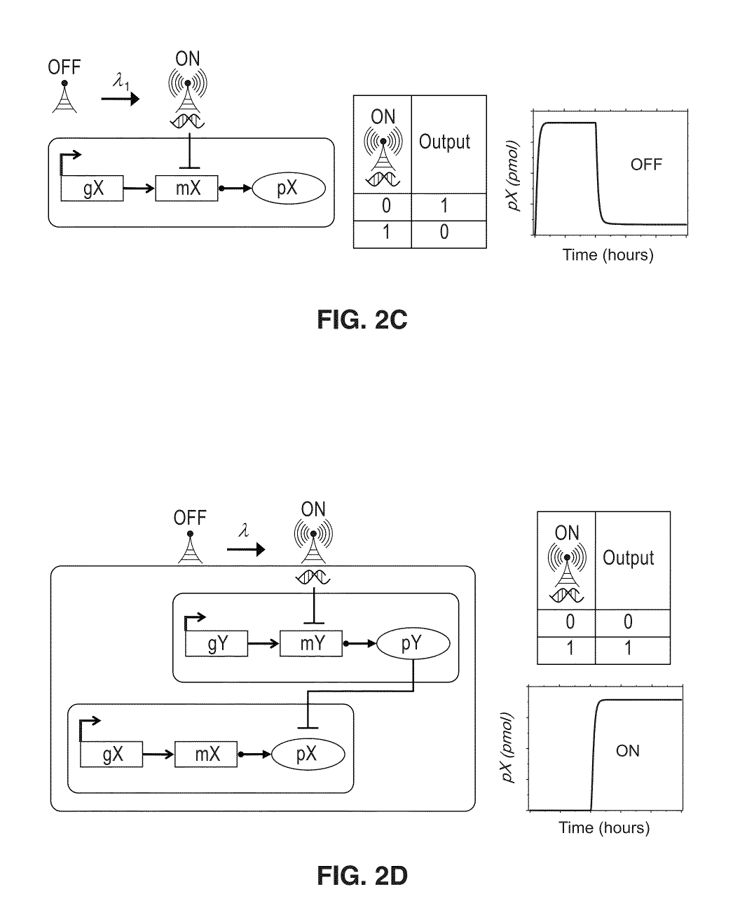

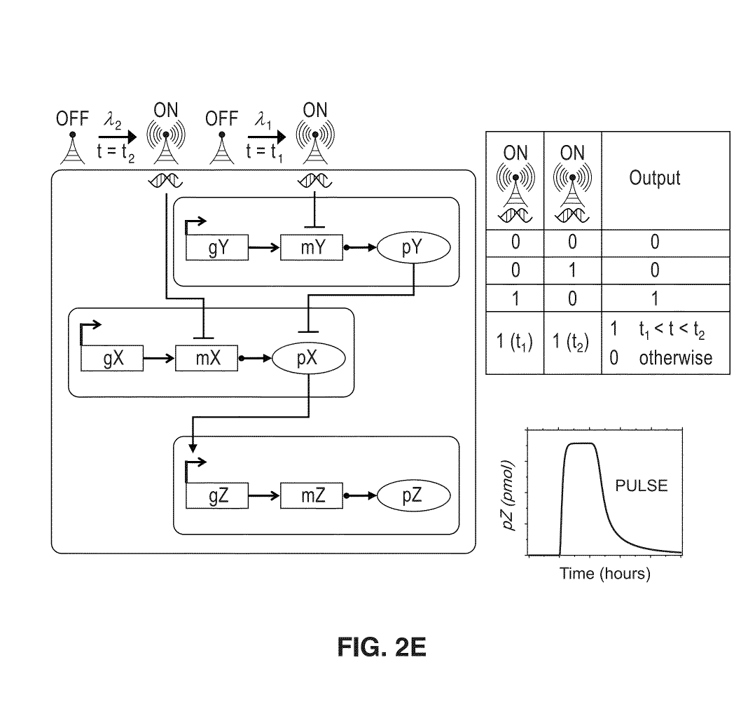

FIG. 2A through FIG. 2E illustrate a photonic gene circuit according to the invention. As shown in FIG. 2A, dynamic optical circuit reconfiguration is enabled by resonant biomolecular nanoantennas as optical inputs to existing circuit connections of living cells, forming photonic gene circuits. The legend notates symbols and circuit connections.

In order to demonstrate the functionality of the biomolecular emitter concept, resonant optical nanoantenna carriers were designed such that absorption cross-sections dominate over its scattering cross-sections in order to efficiently convert absorbed optical energy into surface-localized heat. FIG. 2B illustrates conceptually the function of resonant biomolecular nanoantennas function as selectively addressable optical receivers and biomolecular emitters of molecules such as siRNA. The nanoantenna structure is preferably a nanorod made of gold material and on a scale of approximately 50 μm to 60 μm. The electric field may be calculated using excitation λ=780 nm or λ=650 nm. The antenna effect is “on” at the resonance state and “off” at the non-resonance state of the nanoantenna. The surfaces of the gold nanorods are modified with a cationic phospholipid bilayer and negatively charged siRNA or other molecules are functionalized to the surface, forming the biomolecular nanoantenna carrier. The calculated concentration profiles of siRNA emitted from the biomolecular nanoantenna when the antenna effect is “on” at the resonance state, and no siRNA emitted when the antenna effect is “off” at the non-resonance state is also shown in FIG. 2B.

The heat transfer from the nanoantenna surface to the surrounding cellular environment is highly localized, decaying exponentially within 100 nanometers and therefore is thought to have minimal adverse effects on cells. To further minimize adverse effects, the plasmon resonance wavelength may also be tuned to the NIR since cells are essentially transparent in the NIR regime.

Circuit diagram, logic table, and models of OFF-switch photonic gene circuit, ON-switch photonic gene circuit and PULSE-switch photonic gene circuit are also shown conceptually in FIG. 2C through FIG. 2E. Optically generated, surface-localized heat is used to emit siRNA from the nanoantenna to the cytosol of the cell. The calculated concentration profiles show siRNA is emitted from the nanoantenna when the antenna effect is “on” at the resonance state. This antenna effect can be used to design biomolecular nanoantennas which can be optically addressed to emit siRNA with minimal antenna crosstalk for use in constructing photonic gene circuits.

### Example 2

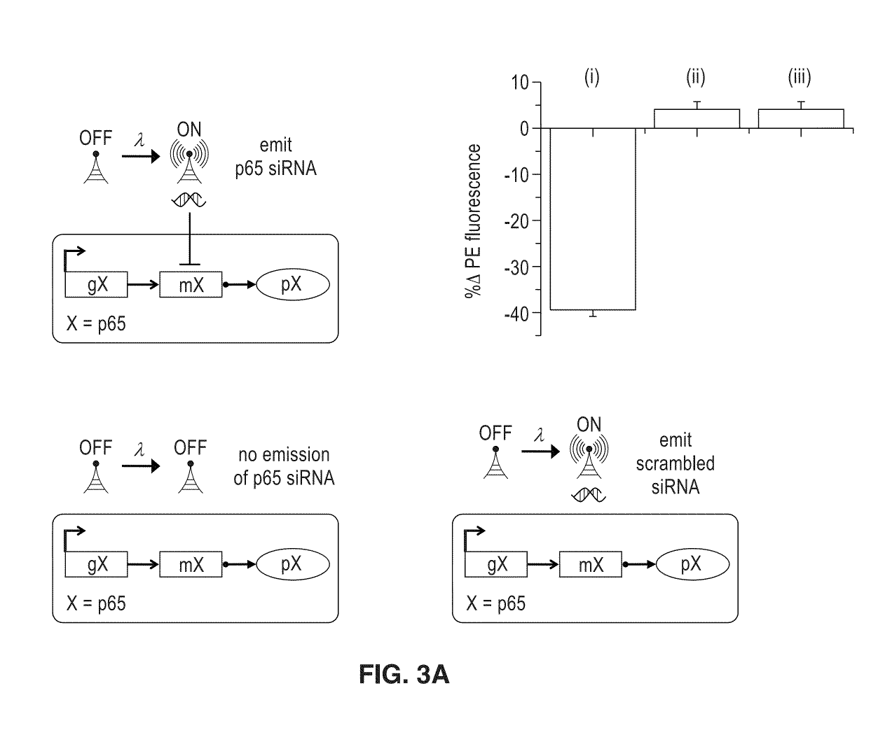

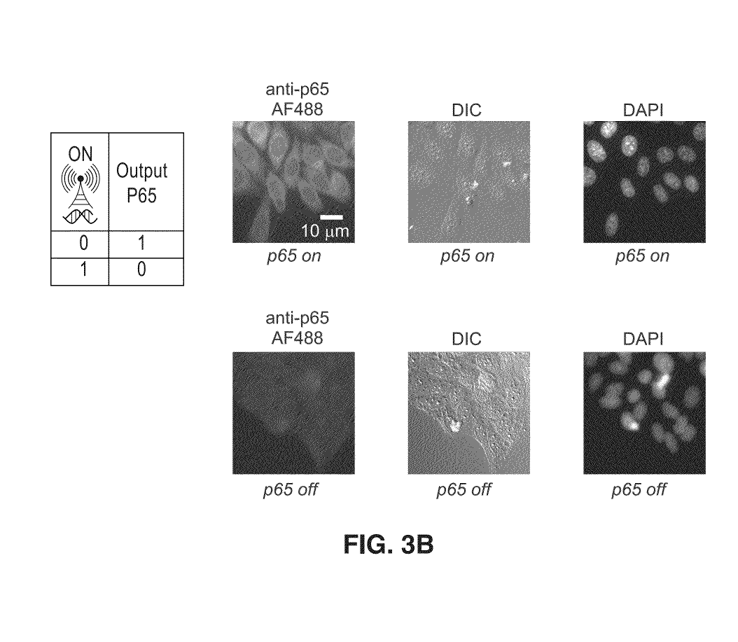

Referring now to FIG. 3A and FIG. 3B, an OFF-switch photonic gene circuit according to the invention is illustrated with a p65 biomolecular nanoantenna. Biomolecular nanoantennas were synthesized and experimentally characterized as functional optical receivers and biomolecular emitters of siRNA. Inside living cells, interfering siRNA can be selected that silences intracellular genes in a highly sequence-specific manner, but alone, it lacks the temporal control necessary for precise modulation. Biomolecular nanoantennas combine the benefits of sequence-specificity with spatiotemporal control. Optical silencing of endogenous genes has been successfully demonstrated using interfering oligonucleotides liberated from nanoantennas. Optical gene silencing was used as an optical input signal to interface existing circuit connections of living cells in order to engineer photonic gene circuits.

The circuit diagram for an OFF-switch photonic gene circuit is shown in FIG. 3A. Gene X (gX) is transcribed into messenger RNA X (mX) which is then translated into protein X (pX). A biomolecular nanoantenna is introduced into the circuit diagram as an optical input. When the biomolecular nanoantenna is optically addressed, siRNA targeting X is emitted from the biomolecular nanoantenna, thereby turning off mX and subsequently pX due to optical gene silencing of X.

To experimentally demonstrate the OFF-switch circuit in HeLa cells, the activated isoform nuclear factor κB-p65 (p65) was chosen to represent X, and flow cytometric analysis was used to quantify the OFF-switch of p65 (i.e. a decrease in p65 protein levels) in single HeLa cells. To demonstrate the OFF-switch photonic gene circuit (p65 off), HeLa cells were obtained and washed once with Optimem media. A 0.5 μL concentrated pellet of biomolecular nanoantennas (2.5 A.R.) functionalized with p65 siRNA was resuspended in 100 μL of Optimem media, gently mixed, and added to each well of the 96-well plate. The cells were allowed to incubate for 4 hours at 37° C. After internalization of biomolecular nanoantennas for 4 hours, the media was replaced with fresh supplemented DMEM culture media. The 96-well plate was placed in a CO₂-filled, sealed container, containing a high transmission glass window (Edmund Optics). Wells were illuminated from a top with 50 mW of 660 nm CW diode laser (Newport Corp.) with a spot size of 2 mm (one quadrant of a well in a 96-well plate) for 15 minutes. After illumination, cells were allowed to incubate for an additional 72 hours at 37° C. Cells were then immunostained for p65, and analyzed by flow cytometry and by immunofluorescence imaging.

Flow cytometric analysis of p65 OFF-switch photonic gene circuit in single HeLa cells is also shown in FIG. 3A. HeLa cells were immunostained using phycoerythrin (PE) labeled anti-p65. Flow cytometric data expressed as percent change of mean PE fluorescence intensity between experiment and reference cells for OFF-switch photonic gene circuit, non-resonant control, and scrambled control. Circuit diagrams for p65 OFF-switch photonic gene circuit; non-resonant control and for scrambled control are also shown in FIG. 3A. Additionally, in FIG. 3B, the logic table for the OFF-switch photonic gene circuit and Immunofluorescence imaging of p65 OFF-switch photonic gene circuit in HeLa cells: DIC, anti-p65-AF488 immunostaining of p65, and DAPI staining of nuclei are shown.

In the scheme of FIG. 3A, it was observed that p65 protein levels decreased by Δ=40% as a result of the OFF-switch photonic gene circuit. No decrease in p65 protein levels was observed when optically addressed, non-resonant biomolecular nanoantennas failed to emit p65 siRNA. No decrease in p65 protein levels were observed when optically addressed biomolecular nanoantennas emitted scrambled siRNA. A logic table summarizing the OFF-switch behavior is shown in FIG. 3B, where “0” (off-state) and “1” (on-state) represent low and high concentrations, respectively. In FIG. 3B, HeLa cells immunostained for p65 showed an overall high fluorescence when the input was “0” and an overall low fluorescence when the input was “1” (optically addressed OFF-switch).

Of course, OFF-switch photonic gene circuits presented in FIG. 3A are not limited to p65 and can be constructed for virtually any protein-of-interest. OFF-switch photonic gene circuits are also modular, and can be combined with other naturally occurring sub-circuits and/or other photonic sub-circuits.

### Example 3

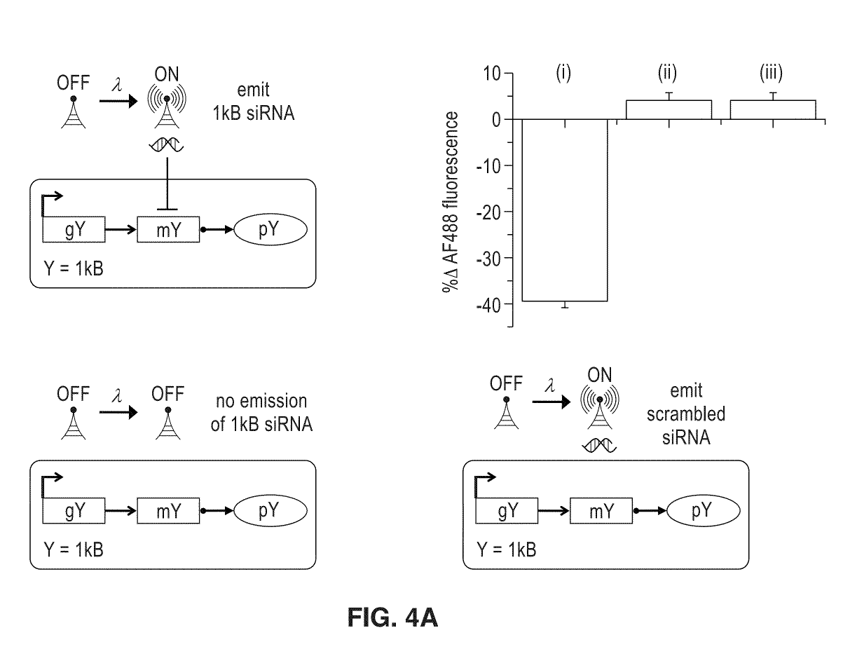

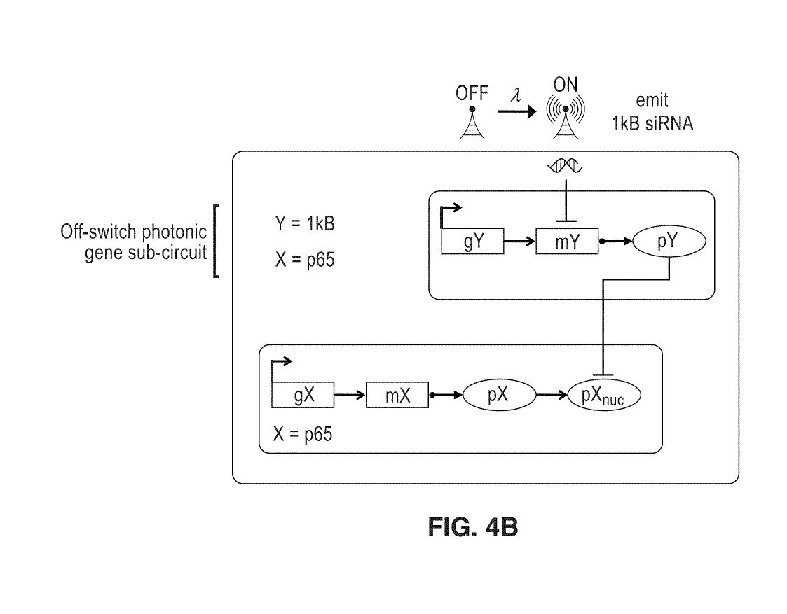

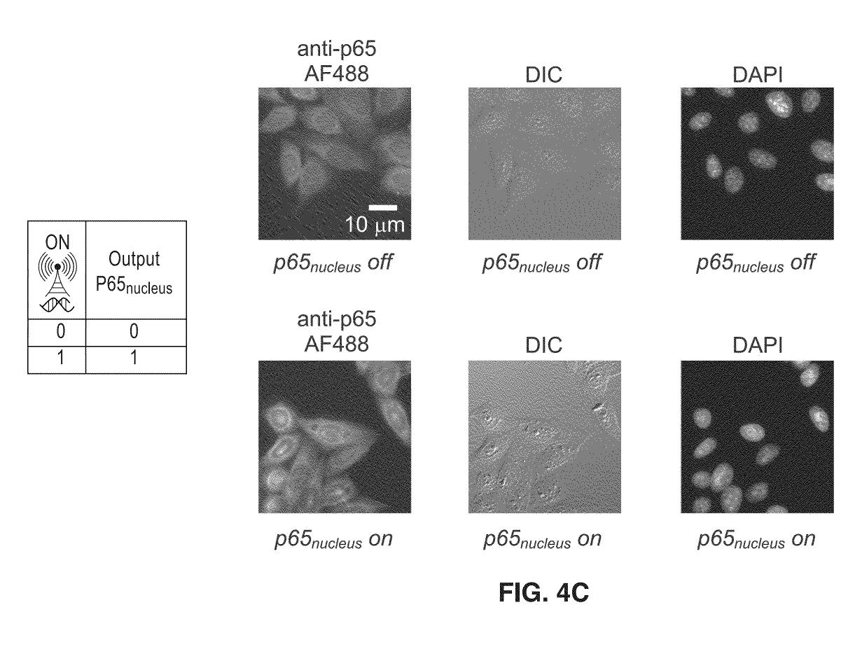

FIG. 4A through FIG. 4C illustrate an ON-switch photonic gene circuit according to the invention constructed using a modular OFF-switch sub-circuit with IκB and p65 chosen to represent Y and X, respectively. FIG. 4A shows a circuit diagram for IκB OFF-switch sub-circuit (i); a circuit diagram for non-resonant control (ii); and a circuit diagram for scrambled control (iii). The flow cytometric analysis of IκB OFF-switch sub-circuit in single HeLa cells. HeLa cells immunostained using AF488 labeled anti-IκB were used. Flow cytometric data is expressed as percent change of mean AF488 fluorescence intensity between experiment and reference cells for IκB OFF-switch sub-circuit, non-resonant control, and scrambled control. FIG. 4C shows a logic table for ON-switch photonic gene circuit. In the on-state, p65 translocates to the nucleus (p65_{nucleus}). Immunofluorescence imaging of ON-switch photonic gene circuit in HeLa cells: DIC, anti-p65-AF488 immunostaining of p65, and DAPI staining of nuclei are also shown.

In the ON-switch photonic gene circuit diagram in FIG. 4B, genes (gX, gY) are transcribed into messenger RNA (mX, mY) and are then translated into proteins (pX, pY), where pY inhibits the active form of pX, represented by pX_{nuc}, by sequestering pX_{nuc} in the cytoplasm (off-state). To construct an ON-switch of pX_{nuc}, a modular OFF-switch sub-circuit was introduced into this circuit diagram to generate a double-negative signal (i.e., inhibit the inhibitor pY). Therefore, when the biomolecular nanoantenna is optically addressed, pY is turned off, thereby enabling pX_{nuc} to turn on and freely translocate to the nucleus (on-state).

To demonstrate the ON-switch photonic gene circuit (p65_{nuc} on), HeLa cells were acquired and washed once with Optimem media. 0.5 μL concentrated pellet of biomolecular nanoantennas (4.0 A.R.) that were functionalized with IκB siRNA was resuspended in 100 μL of Optimem media, gently mixed, and added to each well of the 96-well plate. The cells were allowed to incubate for 4 hours at 37° C. After internalization of biomolecular nanoantennas for 4 hours, the media was replaced with fresh supplemented DMEM culture media. The 96-well plate was placed in a CO₂-filled, sealed container, containing a high transmission glass window (Edmund Optics). Wells were illuminated from the top with a 50 mW of 785 nm CW diode laser (Newport Corp.) and a spot size of 2 mm (one quadrant of a well in a 96-well plate) for 15 minutes. After illumination, cells were allowed to incubate for an additional 72 hours at 37° C. Cells were either immunostained for IκB and analyzed by flow cytometry, or immunostained for p65 and analyzed by immunofluorescence imaging.

To experimentally demonstrate this ON-switch circuit configuration in HeLa cells, p65 was chosen to represent X and the inhibitor κB (IκB) was selected to represent Y. Flow cytometric analysis was used to quantify the IκB OFF-switch sub-circuit (i.e. a decrease in IκB protein levels) in single HeLa cells. In FIG. 4A, IκB protein levels decreased by λ=40% as a result of the OFF-switch sub-circuit. Having confirmed the IκB OFF-switch sub-circuit, the ON-switch of p65_{nuc} was studied using immunofluorescence imaging. A logic table summarizing the ON-switch behavior is shown in FIG. 4B. In the off-state, IκB and p65 form an inactive complex such that p65 is sequestered in the cytoplasm. Therefore, HeLa cells immunostained for p65 showed an overall low fluorescence in the nucleus when the input was “0”. Conversely, HeLa cells showed a strong nuclear presence of p65 (p65_{nuc}) when the input was “1” (optically addressed ON-switch). To locate cells boundaries and nuclei, DIC images and DAPI images were placed adjacent to p65 immunostained images. Similar to OFF-switch circuits, ON-switch photonic gene circuits are also modular and can potentially be combined with multiple sub-circuits to construct larger and more sophisticated photonic gene circuits.

### Example 4

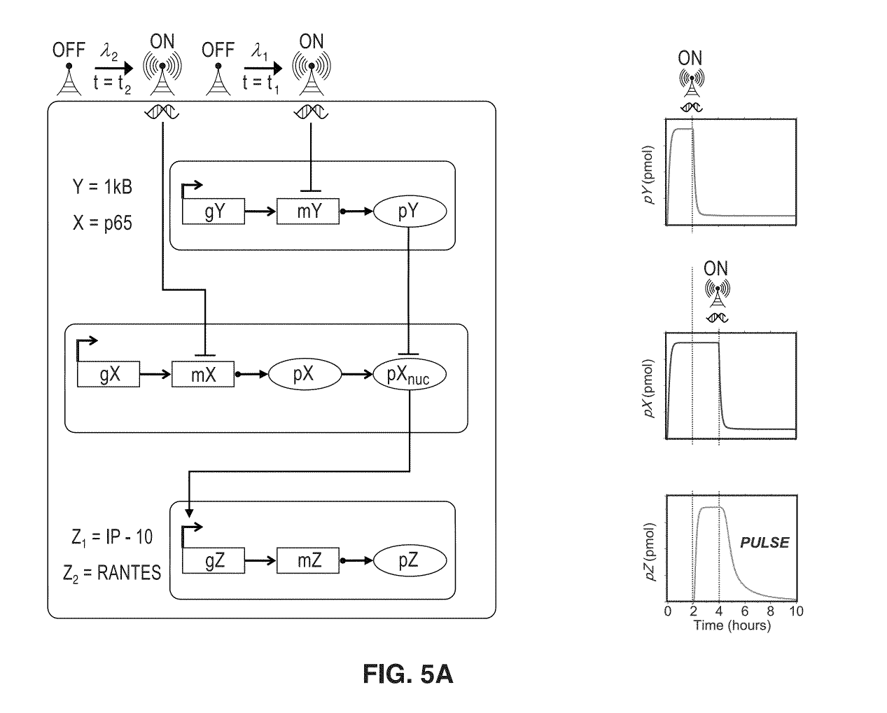

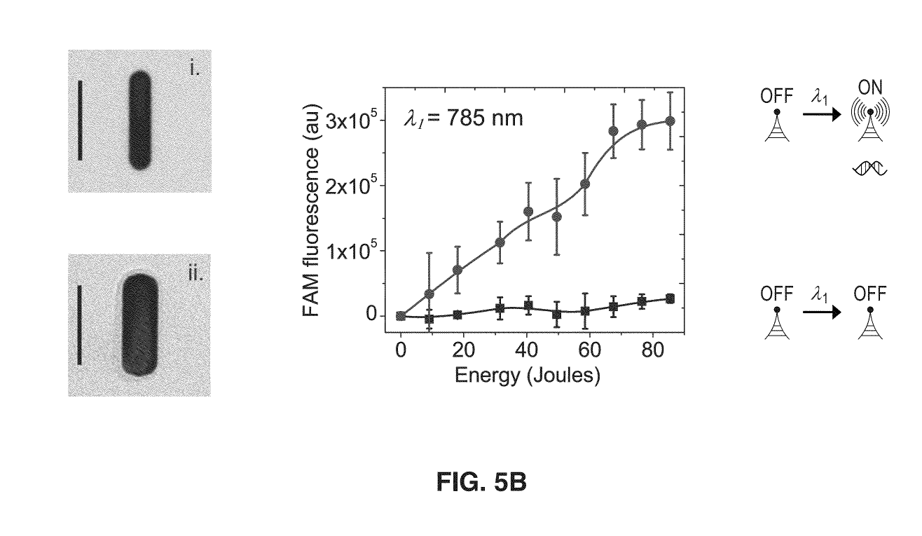

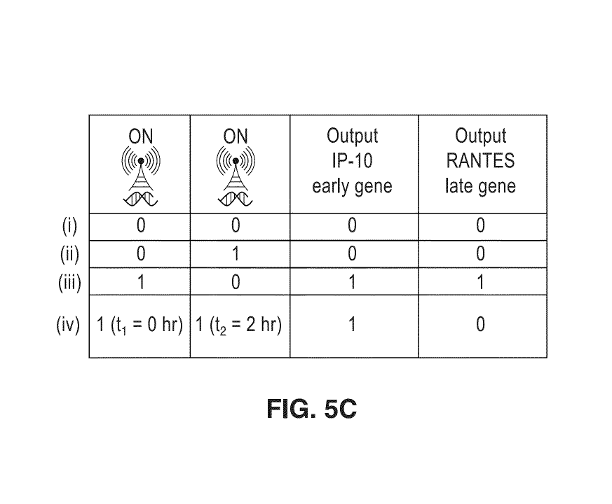

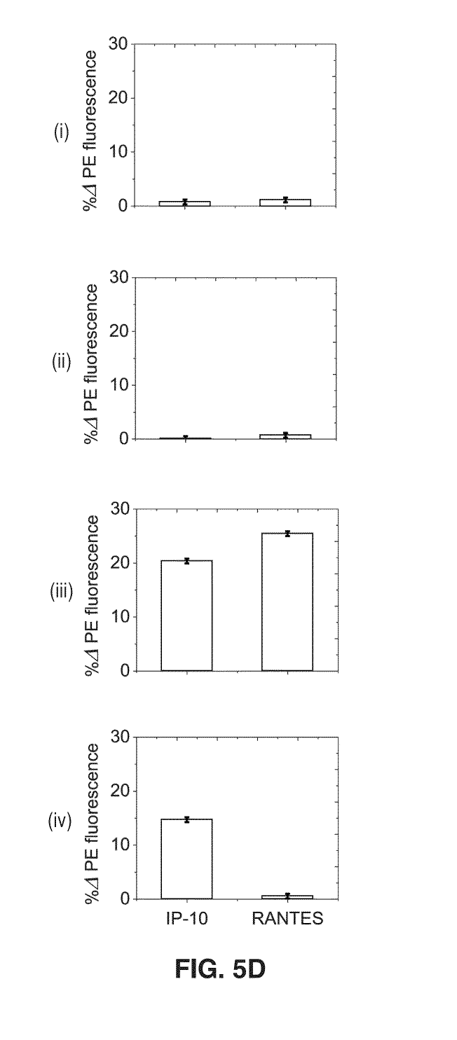

FIG. 5A through FIG. 5D illustrates a PULSE-switch photonic gene circuit according to the invention constructed using multiple, independently operating sub-circuits. To construct larger photonic gene circuits from multiple sub-circuits, modular sub-circuits should function independently of each other. This can be accomplished with biomolecular nanoantennas that operate at distinct optical wavelengths as illustrated in FIG. 5B, based on aspect ratio (A.R.), with minimal antenna crosstalk.

Fluorescence analysis was conducted to measure antenna crosstalk between 4.0 A.R. and 2.5 A.R. biomolecular nanoantennas. A statistically significant increase in fluorescence intensity was seen when optically addressed resonant biomolecular nanoantennas emitted fluorescently labeled siRNA, while no increase in fluorescence intensity was observed when non-resonant nanoantennas were optically addressed as shown in FIG. 5B, indicating minimal antenna crosstalk.

Having confirmed that biomolecular nanoantennas can operate at distinct optical wavelengths with minimal antenna crosstalk, a PULSE-switch photonic gene circuit from multiple, independently operating sub-circuits was constructed. In the circuit diagram of FIG. 5A, ON-switch and OFF-switch sub-circuits are combined to form a PULSE-switch circuit. The ON-switch sub-circuit functions to initiate the pulse of target protein pZ at time t₁. In this ON-switch sub-circuit, biomolecular nanoantennas (4.0 A.R.) are optically addressed at time t₁ using λ₁=785 nm to emit siRNA targeting Y. This causes pY to turn off, pX_{nuc} to turn on, and subsequently target pZ to turn on. The OFF-switch sub-circuit then operates to terminate the pulse of target pZ at time t₂. In this OFF-switch sub-circuit, biomolecular nanoantennas (2.5 A.R.) are optically addressed at time t₂ using λ₂=660 nm to emit siRNA targeting X. This causes pX to turn off and subsequently target pZ to turn off. To experimentally demonstrate this PULSE-switch photonic gene circuit in HeLa cells, IκB and p65 were chosen to represent Y and X, respectively.

Two targets, IP-10 and RANTES, were selected to represent Z (as Z₁ and Z₂, respectively). IP-10, an early response gene, is known to be immediately activated within 30 minutes of stimulation, whereas RANTES, a late response gene, requires more than 3 hours of stimulation to turn on. It was reasoned that if a 2 hour PULSE-switch was constructed, IP-10 should turn on while RANTES should remain off.

To demonstrate the PULSE-switch photonic gene circuit (IP-10 on, RANTES off), acquired HeLa cells were washed once with Optimem media. 0.5 μL concentrated pellet of biomolecular nanoantennas (4.0 A.R.) functionalized with IκB siRNA was added to 0.5 μL concentrated pellet of biomolecular nanoantennas (2.5 A.R.) functionalized with p65 siRNA and was resuspended in 200 μL of Optimem media, gently mixed, and added to each well of the 96-well plate. The cells were allowed to incubate for 4 hours at 37° C. After internalization of biomolecular nanoantennas for 4 hours, the media was replaced with fresh supplemented DMEM culture media. The 96-well plate was placed in a CO₂-filled, sealed container, containing a high transmission glass window (Edmund Optics). Wells were illuminated from the top with 50 mW of 785 nm CW diode laser (Newport Corp.) with a spot size of 2 mm (one quadrant of a well in a 96-well plate) for 15 minutes. Two hours after initial illumination with 785 nm light, wells were illuminated from the top with 50 mW of 660 nm CW diode laser (Newport Corp.) with a spot size of 2 mm (one quadrant of a well in a 96-well plate) for 15 minutes. After illumination, cells were allowed to incubate for an additional 72 hours at 37° C. The media was replaced with fresh supplemented DMEM culture media containing 3 μm monensin (M5273, Sigma) 48 hours after illumination. The cells were allowed to incubate for an additional 24 hours at 37° C. Cells were then immunostained for IP-10 or RANTES and analyzed by flow cytometry.

To implement the 2 hour PULSE-switch, a logic table for IP-10 and RANTES was constructed summarizing four possible conditions as seen in FIG. 5C. The conditions listed in the logic table were shown to function correctly using flow cytometric analysis of IP-10 and RANTES. Flow cytometric analysis of 2 hour PULSE-switch photonic gene circuit in single HeLa cells corresponding to logic table of FIG. 5C is shown in FIG. 5D. HeLa cells were immunostained using phycoerythrin (PE) labeled anti-IP-10 or PE labeled anti-RANTES. Flow cytometric data expressed as percent change of the mean PE fluorescence intensity between experiment (conditions listed in logic table) and reference cells. Notably, the results shown in FIG. 5D (iv), confirmed that IP-10 is turned on while RANTES is turned off as a result of the 2 hour PULSE-switch.

In addition to PULSE-switch photonic gene circuits, other sophisticated circuit configurations can also be conceivably constructed in the future, such as photonic gene oscillators and photonic logic circuits. Strategies for improving signal strength (e.g., siRNA efficiency) can be addressed to improve overall circuit performance. Photonic gene circuits are a promising approach to systematically study native gene circuits in complex, naturally occurring living systems. Since native gene circuits remain genomically unaltered, photonic gene circuits are a promising alternative to synthetic circuits for studying temporal dynamics as they naturally occur in vivo. As a future therapeutic strategy, photonic gene circuits could be used to probe, identify, and reconfigure malfunctioning gene circuits in complex, naturally occurring living systems. Thus, photonic gene circuits will play a pivotal role in engineering functional gene circuits useful for fundamental bioscience, bioengineering, and medical applications are envisioned.

### Example 5

Gold nanorods of aspect ratios 2.5 and 4.0 used in the preceding examples were synthesized by adapting a seed-mediated growth method to an RNase-free environment. All solutions were prepared using 0.2 μM filtered DEPC-treated water. All glassware and metalware were baked at 240° C. for 24 hours to remove exogenous RNases. All pipetting devices and counter space was treated with 70% ethanol. All disposable plastic pipette tips and centrifuge tubes were certified to be free of RNase. The resultant CTAB-coated gold nanoantennas were ensured to be free of RNases by detecting RNase activity over time. RNase activity in the supernatant solution was detected using an RNase activity kit (AM1964, Ambion) and was quantitatively measured over time using a fluorometer (Fluoromax-3, Horiba Jobin Yvon). The concentration of CTAB-coated nanorods was confirmed by adjusting to an absorbance of 1 at the longitudinal plasmon resonance wavelength using UV-VIS spectroscopy (8453, Hewlett Packard). Aspect ratios were determined by scanning electron microscopy and transmission electron microscopy.

For internalization, the nanorods were functionalized with a coating of cationic bilayer of phospholipids to improve entry into living cells. The CTAB surfactant on the surface of the nanorods during synthesis was exchanged with a cationic phospholipid bilayer to form biologically functional cationic phospholipid-gold nanoantennas. In summary, to remove excess CTAB surfactant, 500 μL unmodified CTAB-coated nanoantennas (UV-VIS absorbance of 1) were centrifuged at 5000 rpm for 10 minutes. A 10 μL pellet was transferred to a new microcentrifuge tube, redispersed in 500 μL of nuclease-free water, briefly vortexed, and sonicated for 1 minute. To replace CTAB surfactant with a phospholipid bilayer membrane at the nanoantenna surface, the nanoantennas were centrifuged again at 5000 rpm for 10 minutes. A 10 μL pellet was transferred to a new microcentrifuge tube, resuspended in 50 μL of Oligofectamine, briefly vortexed, and sonicated for 1 minute.

After the CTAB coating was replaced with a cationic phospholipid coating, siRNA was then conjugated to the coated nanoantennas. IκB siRNA was conjugated to nanoantennas (plasmon resonance wavelength 780 nm). p65 siRNA was conjugated to a second type of nanoantenna (plasmon resonance wavelength 650 nm). To 500 μL of nanoantennas solution, 2 μL of 100 μM siRNA was added. The solution was vortexed and allowed to incubate for 30 minutes. To remove excess siRNA from solution, nanoantennas were washed with nuclease-free water by centrifugation at 5000 rpm for 10 minutes and finally resuspended in 500 μL of nuclease-free water. After preparation of siRNA-nanoantennas (i.e., the biomolecular nanoantennas), an absorbance of 0.2 was measured by UV-VIS (8453, Hewlett Packard). By comparing with the original nanoantennas' UV-VIS absorbance of 1, the concentration of biomolecular nanoantennas was estimated to be approximately ⅕ the original concentration (approximately 6 μg/mL or 1.4E11 nanoantennas/mL).

The conjugates were concentrated into a 0.5 μL pellet by centrifugation for cell internalization as described in Example 8.

### Example 6

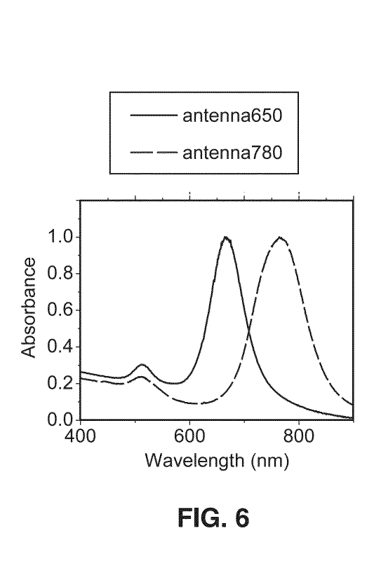

UV-VIS spectra of gold rod-shaped biomolecular nanoantennas with longitudinal plasmon resonances at 780 nm and 650 nm are shown in FIG. 6. Prior to optically addressing biomolecular nanoantennas, the controlled thermal liberation of siRNA from the surface was first demonstrated. If siRNA dissociates from the cationic phospholipid bilayer at elevated temperatures, this unbinding event should change the dielectric constant of the medium locally surrounding the nanoantennas and therefore result in an observable shift in the longitudinal plasmon resonance wavelength of the nanoantennas.

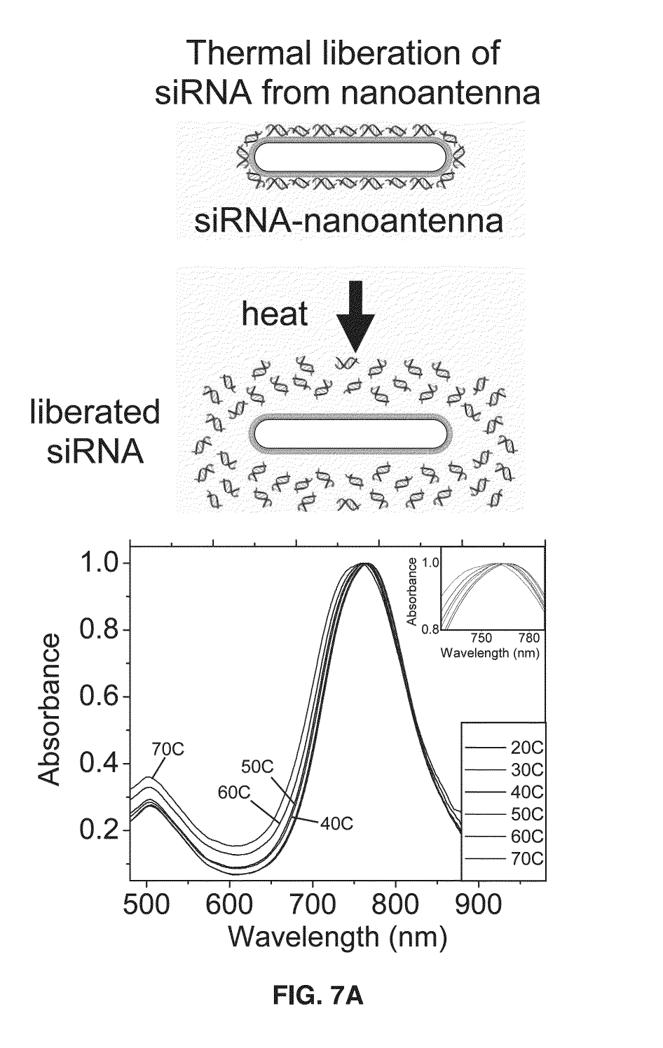

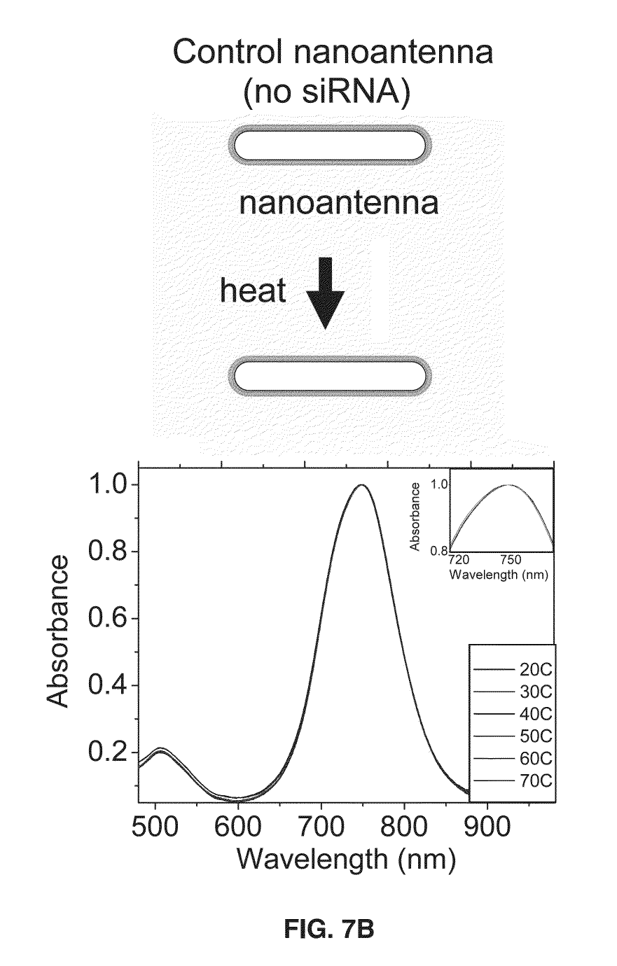

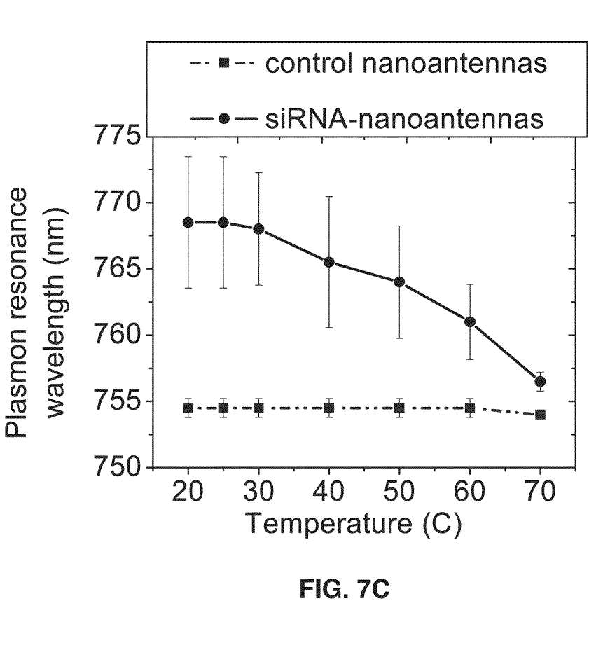

Characterization of thermally liberated siRNA is shown in FIG. 7A through FIG. 7C. FIG. 7A shows UV-VIS spectra of siRNA-nanoantennas as temperature was increased from 20° to 70° C. In contrast, FIG. 7B is a UV-VIS spectrum of control nanoantennas as temperature increased from 20° to 70° C. FIG. 7C shows the longitudinal plasmon resonance wavelength of control nanoantennas and siRNA-nanoantennas as a function of temperature.

A UV-VIS spectrometer containing a thermal-jacketed cell was used to evaluate the temperature-dependent absorbance of the nanoantenna-containing solutions. To each separate cell of an 8-cell micro sample holder (208-92086, Shimadzhu), 50 μL of siRNA-nanoantennas or unconjugated nanoantennas were added. Samples were simultaneously heated from 20° C. to 70° C. (10° C. increment, 5 minutes) using a temperature-controlled UV-VIS spectrometer (2501, Shimadzhu). Absorbance spectra were collected for each sample at each temperature increment.

Control nanoantennas lacking siRNA cargo showed no shift in the longitudinal plasmon resonance wavelength at elevated temperatures as shown in FIG. 7B, indicating that the phospholipid bilayer remained stable at elevated temperatures. In contrast, siRNA-nanoantenna conjugates showed a marked blue-shift in the longitudinal plasmon resonance wavelength at elevated temperatures due to the dissociation of siRNA from the surface (FIG. 7A). Additionally, the longitudinal plasmon resonance wavelength of the siRNA-nanoantennas blue-shifts with incrementing temperature until it eventually matches the longitudinal plasmon resonance wavelength of the control nanoantennas, strongly suggesting the complete dissociation of siRNA from the intact phospholipid bilayer at 70° C. (See FIG. 7C).

### Example 7

Biomolecular nanoantennas optically addressed to selectively liberate siRNA were demonstrated. Having established that siRNA can be thermally dissociated from the cationic phospholipid bilayer, nanoantennas were then optically addressed. Because their narrow longitudinal plasmon resonance bands are spectrally separated as seen in FIG. 6, rod-shaped nanoantennas can selectively receive optical signals at a given frequency and preferentially emit biomolecular information.

To demonstrate, fluorescently-labeled FAM-siRNA was bound to gold nanoantennas and the unbound FAM-siRNA was then removed from the background solution by centrifugation. To a 2 mm path length quartz cuvette (3-2.45-Q-2, Starna Cells Inc.), 50 μL of FAM-siRNA-nanoantennas with 650 nm plasmon resonance wavelength or FAM-siRNA-nanoantennas with 780 nm plasmon resonance wavelength were added. Samples were illuminated from the top with 50 mW of either 785 nm or 660 nm light with a spot size of 2 mm. During illumination, fluorescence emission spectra were collected at 2 minute intervals for each sample using a fluorometer (Fluoromax-3, Horiba Jobin Yvon). A shortpass filter was positioned in front of the fluorometer's detector to block the detector from the laser sources.

An 80 mW 785 nm CW diode laser (Newport Corp.) and a 60 mW 660 nm CW diode laser (Newport Corp.) were co-aligned such that their beams simultaneously overlapped. Firstly, both lasers were positioned parallel to each other on a manually-adjustable xyz stage. A dichroic mirror (Omega Optical) was positioned below the 785 nm laser. A mirror (Omega Optical) was placed below the 660 nm laser. To make the two beams orthogonal to each other, the 660 nm laser beam was reflected 90° towards the direction of the other laser by the mirror. The dichroic mirror transmitted the 785 nm laser beam and reflected the 660 nm laser beam another 90° such that both beams simultaneously overlapped and co-aligned with each other. To circularly polarize the light, an achromatic quarter-wave plate (CVI Laser Corp.) was placed below the co-aligned beams.

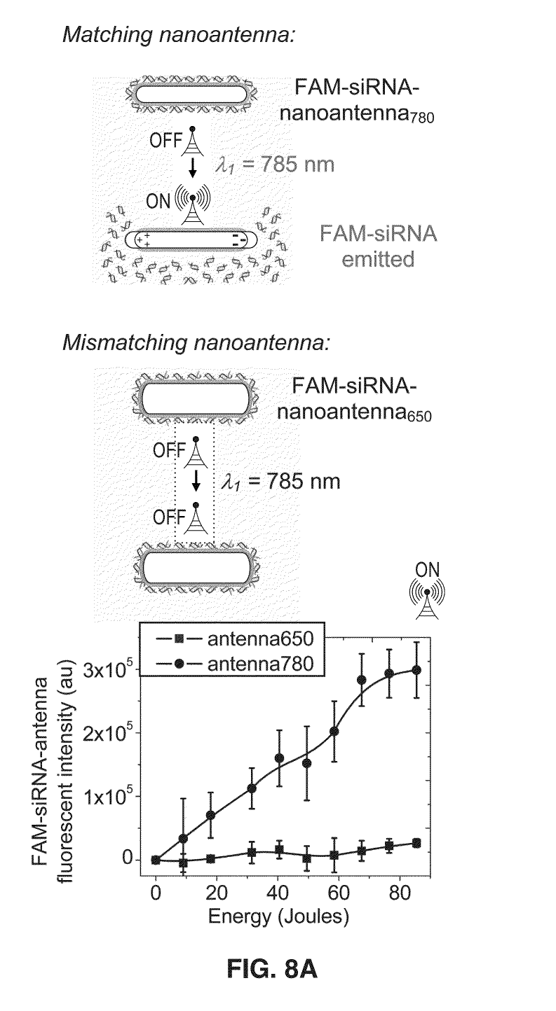

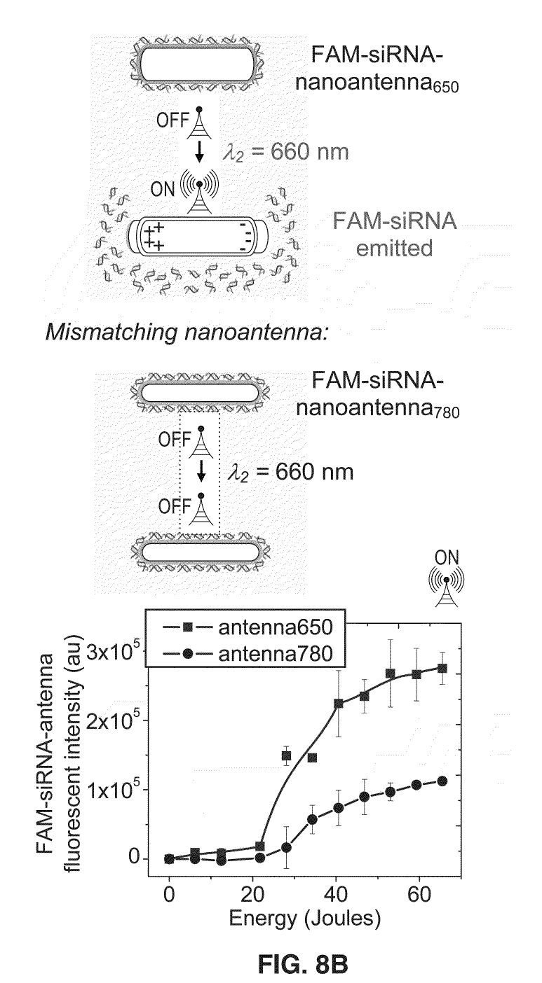

As seen in FIG. 8A, nanoantennas were optically addressed using λ₁=785 nm. Matching nanoantennas emitted FAM-siRNA into solution and the fluorescent intensity was measured. A statistically significant increase in the fluorescent intensity was seen when matching nanoantennas (longitudinal plasmon resonance wavelength 780 nm, aspect ratio 4.0) were addressed. In sharp contrast, no increase in fluorescent intensity was observed when mismatching nanoantennas were addressed, confirming that there was no signal interference with mismatching nanoantennas.

Next, nanoantennas were optically addressed using λ₂=660 nm as illustrated in FIG. 8B. A statistically significant increase in the fluorescent intensity was seen when matching nanoantennas (longitudinal plasmon resonance wavelength 650 nm, aspect ratio 2.5) were addressed, while only a minimal increase in fluorescent intensity was observed when mismatching nanoantennas were addressed. Some signal interference occurred with mismatching nanoantennas, but this can be circumvented by carefully designing nanoantennas to achieve wider spectral separation between their longitudinal plasmon resonance bands.

To calibrate the fluorescent intensity to the concentration of siRNA liberated from nanoantennas carriers into solution, the fluorescent intensities of known concentrations of FAM-siRNA were also measured. Nanoantennas of concentration 1E11 nanoantennas/mL based on UV-VIS measurements were observed to have released approximately 0.05 μM siRNA after illumination. As a positive control, Triton X-100 detergent was used to disrupt the cationic phospholipid bilayer around nanoantennas, thereby ensuring the complete liberation of all bound siRNA from nanoantennas into solution. The concentration of siRNA released using Triton X-100 matched closely to the concentration of optically liberated siRNA, strongly suggesting complete liberation of siRNA from optically addressed nanoantennas.

### Example 8

Internalization of biomolecular nanoantennas in tissue culture cells was then demonstrated. The human cervical carcinoma cell line HeLa was purchased from the American Type Culture Collection (ATCC). Dulbecco's modified eagle's media (DMEM) formulated with high glucose and GlutaMAX was purchased from Invitrogen and was supplemented with 10% heat-inactivated fetal bovine serum. Cells were seeded at an initial concentration of 20,000 cells/well in a 96-well plate, cultured in the supplemented media, and maintained in a 37° C. incubator with 5% CO₂ humidified air.

Visualization of Internalized Biomolecular Nanoantennas in HeLa Cells. Biomolecular nanoantennas were internalized into HeLa human cervical cancer cells. For visualization purposes, HeLa cells were seeded onto 12 mm gridded glass coverslips (72265-12, Electron Microscopy Sciences) at 30,000 cells/well in a 24-well plate for 24 hours before use. HeLa cells were then washed once with Optimem media. The 0.5 μL concentrated pellet of biomolecular nanoantennas was resuspended in 100 μL of Optimem media, gently mixed, and added to each well of the 24-well plate. The cells were allowed to incubate for 4 hours at 37° C. Cells were then fixed by incubating cells with 2% paraformaldehyde (15712-S, Electron Microscopy Sciences) in 1×PBS per well for 10 minutes. Cell nuclei were stained with DAPI (D-1306, Invitrogen) by incubating cells in 300 nM DAPI in 1×PBS per well for 5 minutes. 1×PBS was used to twice-wash the cells. The coverslip containing fix, adhered cells was then placed facedown and adhered to a microscope slide. Cells were located using the grids imprinted on the coverslips.

Darkfield microscopy was used to visualize internalized biomolecular nanoantennas. Darkfield scattering was visualized using an inverted microscope (Axiovert, Zeiss) at 40× magnification. Broadband white light was shined onto the adhered cells from an oblique angle using a darkfield condenser lens. The scattered light alone was collected using a microscope objective lens with a numerical aperture (NA) of 0.65 that was smaller than the NA (1.2-1.4) of the illumination condenser lens. To locate cells' boundaries and nuclei, DIC images were overlaid with DAPI-stained images and placed adjacent to darkfield scattering images. DIC and DAPI were visualized using an upright fluorescence microscope (Axio Imager, Zeiss) at 40× magnification.

Scattered light from cells containing nanoantennas was easily differentiated from cells lacking nanoantennas. Long-term viability/cytotoxicity and proliferation studies were previously conducted to ensure that internalized nanoantennas and optical excitation caused no adverse effects on cell behavior.

To estimate the amount of siRNA liberated from nanoantennas into the intracellular space, fluorescently-labeled FAM-siRNA were bound to nanoantennas and unbound FAM-siRNA were then removed from the background solution by centrifugation. Known concentrations of FAM-siRNA-nanoantennas (0, 7E11, 4E11, and 3E11 nanoantennas/mL based on UV-VIS measurements) were internalized in HeLa cells. Fluorescent intensities of individual cells were then measured by flow cytometry and a standard concentration curve of internalized FAM-siRNA-nanoantennas was constructed. Fluorescence quenching by nanoantennas was not observed. To correlate the fluorescent intensities to FAM-siRNA concentration, control cells were incubated with known concentrations of FAM-siRNA (50 nM, 100 nM, and 200 nM) for 5 hours. A standard concentration curve of internalized FAM-siRNA was then constructed based on flow cytometry analysis. These standard curves were utilized to estimate the concentration of siRNA-conjugated nanoantennas (biomolecular nanoantennas) necessary for optical gene silencing.

### Example 9

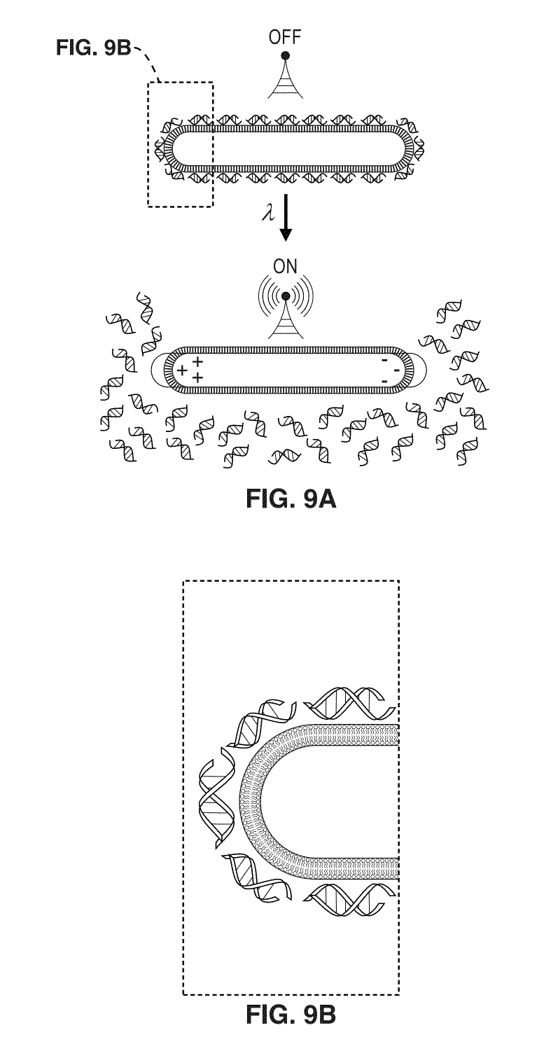

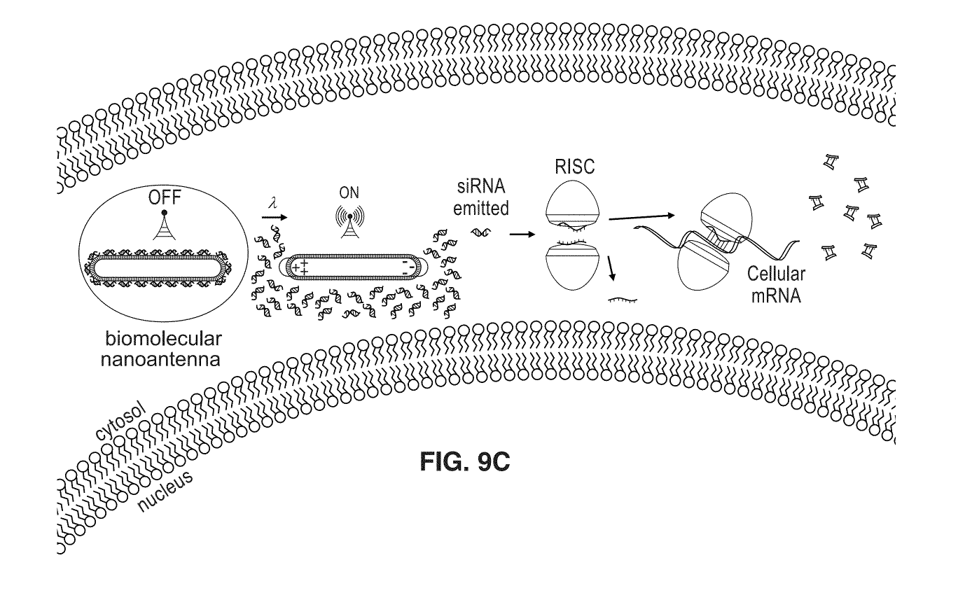

The mechanism of optical gene silencing with siRNA duplexes electrostatically attached to the cationic phospholipid bilayer of the nanoantenna is illustrated in FIG. 9A-C. Interfering siRNA enable sequence-specific silencing of intracellular genes, but alone, lack the temporal control necessary for precise manipulation. While attached to these nanoantenna carriers, siRNA is in an inactive state as shown in FIG. 9A and FIG. 9B. When the excitation wavelength is matched to the plasmon resonance wavelength, the absorbed optical energy is converted to thermal energy, the electrostatic interaction is destabilized, and siRNA molecules are emitted from the nanoantennas.

In living cells, optically addressed nanoantennae photothermally disrupt encapsulating endosomes, enabling unbound siRNA to enter the cytosol. The unbound siRNA then triggers the RNA-induced silencing complex (RISC) to unwind oligonucleotide duplexes, bind to complementary messenger RNA (mRNA), and silence or otherwise influence gene expression. Thus, nanoantennas conjugated with interfering siRNA (i.e. biomolecular nanoantennas) combine the benefits of sequence-specificity and spatiotemporal control for precise manipulation of gene expression. Endogenous genes have been shown to be optically silenced using interfering oligonucleotides liberated from nanoantennas. In this work, we utilize optical gene silencing as an “input signal” to interface existing internal connections of a living cell in order to construct photonic gene circuits.

One embodiment of the on-demand gene silencing using siRNA is shown in FIG. 9C in five steps: Step 1, siRNA duplexes electrostatically attached to nanoantennas and are internalized in cells. Step 2, the nanoantenna is optically addressed and siRNA is emitted from the nanoantenna. Step 3, unbound siRNA triggers cytosolic RISC to unwind the duplex. Step 4, cytosolic RISC binds single-stranded RNA to its complementary mRNA. Step 5, the gene-of-interest is silenced.

### Example 10

For a positive control (siRNA alone), conventional lipofection using Lipofectamine 2000 (11668-019, Invitrogen) was carried out according to manufacturer's instructions. Briefly, HeLa cells were washed once with Optimem media. Cells were then incubated with 25 nM siRNA (Qiagen) in Optimem media for 6 hours. After 6 hours, the media was replaced with fresh supplemented DMEM culture media. Cells were allowed to incubate for an additional 72 hours at 37° C.

### Example 11

For immunostaining for flow cytometry analysis, fluorescently-labeled antibodies recognizing p65 (sc-8008-PE) and IκB (sc-1643-AF488, sc-945-AF488) were purchased from Santa Cruz Biotechnologies. Fluorescently-labeled normal mouse isotype antibodies (sc-2866, sc-3890, and sc-45068) were also purchased from Santa Cruz Biotechnologies and were used as negative controls. Fluorescently-labeled antibodies recognizing IP-10 (IC266P) and RANTES (IC278P), and the appropriate isotype control (IC002P) were purchased from R&D Systems. Permeabilization buffer, for use with p65 and IκB antibodies, was prepared by adding 0.1% (w/v) saponin, 0.3% (w/v) Triton-X, and 0.1% (w/v) NaN₃ to Hank's Balanced Salt Solution (Invitrogen). Permeabilization buffer, for use with IP-10 and RANTES antibodies, was prepared by adding 0.1% (w/v) saponin and 0.06% (w/v) NaN₃ to Hank's Balanced Salt Solution (Invitrogen).

Cells were harvested, resuspended in 250 μL of 1×PBS, and fixed with 250 μL of 4% paraformaldehyde for 10 minutes. After 10 minutes, excess paraformaldehyde was removed by centrifuging and resuspending cells in 400 μL of permeabilization buffer (repeated twice). Cells were then counted to ensure all samples contained the same number of cells prior to immunostaining. For 50,000 cells, 10 μL of antibody or isotype antibody were added. Cells were gently mixed and incubated at room temperature for 45 minutes. After 45 minutes, excess antibodies were removed by centrifuging and resuspending cells in 400 μL of permeabilization buffer. To remove permeabilization buffer, cells were finally centrifuged and resuspended in 500 μL of 1×PBS. LSRII flow cytometer (BD Biosciences) and FlowJo software (Tree Star, Ashland, Oreg.) were used to analyze samples.

Immunostaining of p65 for immunofluorescence Imaging. 72 hours after optical gene silencing of IκB or p65, cells (adhered to glass coverslips) were fixed in cold 50% methanol for 3 minutes on ice followed by cold 100% methanol for 15 minutes on ice. For three times, cells were washed with and incubated in 1×PBS for 5 minutes on a rocker at speed approximately 100 rpm. Cells were blocked with 5% normal mouse serum (01-6501, Invitrogen) in 1×PBS for 30 minutes on a rocker at speed of approximately 100 rpm. For two times, cells were washed with and incubated in 1×PBS for 5 minutes on a rocker at a speed of approximately 100 rpm. Cells were then incubated in 300 nM DAPI (D1306, Invitrogen) in 1×PBS for 5 minutes in the dark on a rocker at a speed approximately 100 rpm. Cells were washed with and incubated in 1×PBS for 5 minutes on a rocker at a speed of approximately 100 rpm. To 200 μL of 1×PBS, 60 μL of anti-p65 (sc8008 AF488, Santa Cruz Biotechnologies) was added. Cells were allowed to incubate for 2 hours in the dark on a rocker at a speed of approximately 100 rpm. For three times, cells were washed with and incubated in 1×PBS for 5 minutes on a rocker at a speed of approximately 100 rpm. A coverslip containing fixed, stained adhered cells was finally placed face down on a microscope slide, sealed, and imaged using fluorescence microscopy.

### Example 12

A finite element method (COMSOL Multiphysics software) was used to model a 3-D gold nanorod (4.0 and 2.5 A.R.) suspended in water and to achieve a solution to the Helmholtz wave equation:

∇×(μᵣ⁻¹∇×E)−k₀²(∈ᵣ−jσω∈₀)E=0.

The gold nanorod was constructed using a cylinder with hemispheres on each end of the cylinder. The relative permeability of gold was assumed to be μᵣ=1 and the complex permittivity of gold ∈ᵣ was assumed to be a function of wavelength λ. A spherical perfectly matched layer and an integration layer, modeled by concentric spheres, were used to reach perfect absorption at the outer boundary and minimize spurious reflections. A plane wave was used for excitation (λ=780 nm or λ=650 nm). The adaptive mesh was refined until the maximum electric field converged.

Modeling of Optically Generated Surface Localized Heat was based on the solution of the bioheat transfer equation:

$$ρ C \frac{\partial T}{\partial t} + ∇ \cdot ( - k ∇ T ) = {ρ}_{b} {C}_{b} {ω}_{b} ( {T}_{b} - T ) + {Q}_{met} + {Q}_{ext} .$$

Time-average resistive heat (W/m³) was used for the spatial heat source Qₑₓₜ under the assumption that electromagnetic energy was converted to heat by resistive heating. The thermal conductivity of gold, density of gold, and specific heat of gold were assumed to be k_{g}=320 W/m-K, ρ_{g}=19300 kg/m³, and C_{g}=129 J/kg-K, respectively. The thermal conductivity of water, density of water, and specific heat of water were assumed to be k_{w}=0.61 W/m-K, ρ_{w}=1000 kg/m³, and C_{w}=42000 J/kg-K, respectively. The metabolic heat source Qₘₑₜ and the perfusion rate ω_{b} were assumed to be insignificant.

Modeling of Biomolecular Emitter. To investigate biomolecular emission, the following thermal diffusion equation was solved:

$$\frac{\partial c}{\partial t} = ∇ \cdot [ D ∇ c + {D}_{T} c ∇ T ] .$$

Electromagnetic energy was assumed to be converted to surface localized heat and was used here for temperature T. It was assumed that siRNA dissociated from the surface of the nanoantenna at elevated temperatures (experimentally demonstrated in sections 3 and 4), and mass flux occurred due to the temperature gradient in addition to the siRNA concentration gradient. The diffusion coefficient and the thermal diffusion coefficient of siRNA were estimated to be to be D=36.00×10⁸ cm²/s and D_{T}=0.45×10⁸ cm²/s-K, respectively, based on double-stranded DNA of similar length. Using the surface density (9.0×10¹² molecules/cm²) and the size dimensions, the initial concentrations of siRNA were calculated to be c₀=1.81×10⁻¹⁴ mol/m³ for 4.0 A.R. nanoantennas and c₀=2.11×10⁻¹⁴ mol/m³ for 2.5 A.R. nanoantennas, respectively.

Modeling of Photonic OFF-switch, ON-switch, PULSE-switch gene circuits. The OFF-switch (Eq. 1-3), ON-switch (Eq. 4-8), and PULSE-switch (Eq. 9-17) circuit configurations were modeled (Matlab software). Genes (gX, gY, gZ) were transcribed into messenger RNA (mX, mY, mZ) which were then translated into proteins (pX, pY, pZ), where gX, gY, gZ, mX, mY, mZ, pX, pY, and pZ denote concentrations. Degradation of messenger mRNA by optical gene silencing using biomolecular nanoantennas functionalized with siRNA (NAsirna1, NAsirna2) were modeled based on Michaelis-Menton/Hill kinetics.

### OFF-Switch Circuit

$$\begin{matrix}\frac{d mX}{d t} = {k}_{X 1} \cdot gX - {k}_{d\_mX} \cdot mX - {k}_{d\_sirna 2 \_mrna} \cdot {β}_{X} \cdot ( \frac{{NA}_{on} sirna 2}{{K}_{x} + {NA}_{on} sirna 2} ) \cdot mX & Equation ( 1 ) \\ \frac{d pX}{d t} = {k}_{X 2} \cdot mX - {k}_{d\_pX} \cdot pX & Equation ( 2 ) \\ \frac{d {NA}_{on} sirna 2}{d t} = {k}_{L 2} * NAsirna 2 - {k}_{d\_sirna 2} * {NA}_{on} sirna 2 & Equation ( 3 )\end{matrix}$$

### ON-Switch Circuit

$$\begin{matrix}\frac{d mY}{d t} = {k}_{Y 1} \cdot gY - {k}_{d\_mY} \cdot mY - {k}_{d\_sirna 1 \_mrna} \cdot {β}_{Y} \cdot ( \frac{{NA}_{on} sirna 1}{{K}_{Y} + {NA}_{on} sirna 1} ) \cdot mY & Equation ( 4 ) \\ \frac{d mX}{d t} = {k}_{X 1} \cdot gX - {k}_{d\_mX} \cdot mX & Equation ( 5 ) \\ \frac{d pX}{d t} = {k}_{X 2} \cdot mX - {k}_{d\_pX} \cdot pX & Equation ( 6 ) \\ \frac{d {pX}_{nuc}}{d t} = {k}_{X 3} \cdot pX - {k}_{d\_pXnuc} \cdot {pX}_{nuc} - {k}_{d\_pXnuc \_pY} \cdot pY \cdot {pX}_{nuc} & Equation ( 7 ) \\ \frac{d {NA}_{on} sirna 1}{d t} = {k}_{L 1} * NAsirna 1 - {k}_{d\_sirna 1} * {NA}_{on} sirna 1 & Equation ( 8 )\end{matrix}$$

### PULSE-Switch Circuit

$$\begin{matrix}\frac{d mY}{d t} = {k}_{Y 1} \cdot gY - {k}_{d\_mY} \cdot mY - {k}_{d\_sirna 1 \_mrna} \cdot {β}_{Y} \cdot ( \frac{{NA}_{on} sirna 1}{{K}_{Y} + {NA}_{on} sirna 1} ) \cdot mY & Equation ( 9 ) \\ \frac{d pY}{d t} = {k}_{Y 2} \cdot mY - {k}_{d\_pY} \cdot pY & Equation ( 10 ) \\ \frac{d mX}{d t} = {k}_{X 1} \cdot gX - {k}_{d\_mX} \cdot mX - {k}_{d\_sirna 2 \_mrna} \cdot {β}_{X} \cdot ( \frac{{NA}_{on} sirna 2}{{K}_{x} + {NA}_{on} sirna 2} ) \cdot mX & Equation ( 11 ) \\ \frac{d pX}{d t} = {k}_{X 2} \cdot mX - {k}_{d\_pX} \cdot pX & Equation ( 12 ) \\ \frac{d {pX}_{nuc}}{d t} = {k}_{X 3} \cdot pX - {k}_{d\_pXnuc} \cdot {pX}_{nuc} - {k}_{d\_pXnuc \_pY} \cdot pY \cdot {pX}_{nuc} & Equation ( 13 ) \\ \frac{d mZ}{d t} = {k}_{Z 1} * gZ * {β}_{Z} * ( \frac{{pX}_{nuc}^{n}}{{K}_{Z} + {pX}_{nuc}^{n}} ) - {k}_{d\_mZ} * mZ & Equation ( 14 ) \\ \frac{d pZ}{d t} = {k}_{Z 2} \cdot mZ - {k}_{d\_pZ} \cdot pZ & Equation ( 15 ) \\ \frac{d {NA}_{on} sirna 1}{d t} = {k}_{L 1} * NAsirna 1 - {k}_{d\_sirna 1} * {NA}_{on} sirna 1 & Equation ( 16 ) \\ \frac{d {NA}_{on} sirna 2}{d t} = {k}_{L 2} * NAsirna 2 - {k}_{d\_sirna 2} * {NA}_{on} sirna 2 & Equation ( 17 )\end{matrix}$$

Kinetic rates and initial values are listed in Table 1 and were approximated similarly to those previously reported in literature for a eukaryotic model. It was assumed no siRNA was emitted by non-resonant biomolecular nanoantennas. It was also assumed that when biomolecular nanoantennas were optically addressed with the correct optical wavelength, all siRNA was emitted. Since the model's timescale was on the order of hours, it was assumed that nanoantennas did not degrade in this timeframe and therefore the concentration of nanoantennas remained the same. Due to the tight packing of siRNA on the nanoantennas, steric hindrances inhibited nuclease degradation of siRNA while siRNA was bound to the nanoantennas. Once siRNA was emitted from nanoantennas, the siRNA degraded with degradation rates as reported in previous literature.

From the discussion above it will be appreciated that the invention can be embodied in various ways, including the following:

1. A method for reconfiguring gene circuits in a cell, comprising: introducing plasmon resonant nanoparticles functionalized with a cationic lipid coating and adsorbed biomolecules into cells; and exposing the cells to light with a wavelength matching a plasmon resonance wavelength of the nanoparticles to release the adsorbed biomolecules from the functionalized nanoparticles into the cells; wherein the released biomolecule influences genetic circuits of the cell.

2. The method as recited in embodiment 1, further comprising: introducing a second plasmon resonant nanoparticle functionalized with a cationic lipid coating and adsorbed biomolecules of a second biomolecule into cells; and exposing the cells to light with a wavelength matching a plasmon resonance wavelength of the second nanoparticles to release the adsorbed second biomolecules from the second functionalized nanoparticles into the cells; wherein the second nanoparticle has a plasmon resonance wavelength that is different than any other nanoparticle plasmon resonance wavelength of nanoparticles introduced into the cell.

3. The method as recited in any of the previous embodiments, further comprising: introducing a third plasmon resonant nanoparticle functionalized with a cationic lipid coating and adsorbed biomolecules of a third biomolecule into cells; and exposing the cells to light with a wavelength matching a plasmon resonance wavelength of the third nanoparticles to release the adsorbed third biomolecules from the third functionalized nanoparticles into the cells; wherein said third nanoparticle has a plasmon resonance wavelength that is different than any other nanoparticle plasmon resonance wavelength of nanoparticles introduced into the cell.

4. The method as recited in any of the previous embodiments, wherein the biomolecule is selected from the group of biomolecules consisting essentially of transcription factor proteins, RNA, DNA and oligonucleotides.

5. The method as recited in any of the previous embodiments, wherein the biomolecule is a small interfering RNA (siRNA) biomolecule.

6. The method as recited in any of the previous embodiments, wherein said lipid coating comprises a cationic phospholipid bilayer coating.

7. The method as recited in any of the previous embodiments, wherein the nanoparticle is a nanorod with an aspect ratio between 2.0 and 8.0.

8. The method as recited in any of the previous embodiments, wherein the nanoparticle is a nanorod with a plasmon resonance wavelength in the near infrared (NIR) spectrum.

9. The method as recited in any of the previous embodiments, wherein the first, second or third biomolecule are the same biomolecule.

10. The method as recited in any of the previous embodiments, further comprising: introducing the nanoparticles simultaneously into the cell; and temporally controlling the light exposure and release of the biomolecules from the nanoparticles in the cell.

11. The method as recited in any of the previous embodiments, further comprising: acquiring one or more groups of small interfering RNA (siRNA) biomolecules, each siRNA group configured to interfere with transcription of a different gene in a cell; and adhering the different groups of siRNA biomolecules onto coated nanoparticles that have a single plasmon resonant wavelength; wherein groups of different siRNA biomolecules are released with a single light exposure.

12. A method for reconfiguring gene circuits in a cell, comprising: providing groups of plasmon resonant nanoparticles, each group of nanoparticles having a discrete plasmon resonance wavelength; coating the groups of nanoparticles with a cationic lipid coating; reversibly coupling different biomolecules with each group of coated nanoparticles to form groups of addressable biomolecular nanoantennas; depositing the biomolecular nanoantennas into cells; and exposing the cells to light with a wavelength matching a plasmon resonance wavelength of each group of nanoparticles to release the coupled biomolecules from the nanoantennas; wherein the released biomolecule influences genetic circuits of the cell; and wherein the release of each group of biomolecules is temporally controlled by the timing of light exposure.

13. The method as recited in any of the previous embodiments, wherein the lipid coating comprises a cationic phospholipid bilayer coating.

14. The method as recited in any of the previous embodiments, wherein the nanoparticles are nanorods with an aspect ratio of between 2.0 and 8.0.

15. The method as recited in any of the previous embodiments, wherein the nanoparticle is a nanorod with a plasmon resonance wavelength in the near infrared (NIR) spectrum.

16. The method as recited in any of the previous embodiments, wherein said biomolecules are selected from the group of biomolecules consisting essentially of transcription factor proteins, RNA, DNA and oligonucleotides.

17. The method as recited in any of the previous embodiments, further comprising: selecting a gene circuit for manipulation; selecting biomolecules that will initiate or terminate the transcription of genes in the selected gene circuit; and providing the selected biomolecules for adsorption with coated nanoparticles.

18. The method as recited in any of the previous embodiments, further comprising: selecting biomolecules that will increase or decrease the rate of transcription of genes in the selected gene circuit.

19. A biomolecule carrier for introducing biomolecules into a cell, comprising: a plasmonic nanoparticle with a plasmon resonant wavelength; and a cationic lipid coating on the exterior surfaces of the nanoparticle.

20. The biomolecule carrier as recited in embodiment 19, wherein the lipid coating comprises a cationic phospholipid bilayer coating.

21. A biomolecule carrier as recited in any of the previous embodiments, wherein the nanoparticles are nanorods with an aspect ratio of between 2.0 and 8.0.

22. A biomolecule carrier as recited in any of the previous embodiments, wherein the nanoparticle is a nanorod with a plasmon resonance wavelength in the near infrared (NIR) spectrum.

Embodiments of the present invention may be described with reference to flowchart illustrations of methods and systems according to embodiments of the invention, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art. In the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112 unless the element is expressly recited using the phrase “means for.”

TABLE 1
Parameter Values For Photonic Gene Circuit Models

| Parameter | Value | |
|---|---|---|
| Rate of mRNA synthesis (k_{X1}, k_{Y1}, k_{Z1}) | 1 | pmol/day |
| Rate of protein synthesis (k_{X2}, k_{Y2}, k_{Z2}) | 100000 | pmol/day |
| Half life of eukaryotic mRNA (τ_{1/2}) | 1 hr = 0.042 day | |
| Half life of eukaryotic protein (τ_{1/2}) | 2 hrs = 0.083 day | |
| Rate of mRNA degradation (k_{d}_{_{—}}_{mX}, k_{d}_{_{—}}_{mY}, k_{d}_{_{—}}_{mZ}) | 16.5 | days⁻¹ |
| Rate of protein degradation (k_{d}_{_{—}}_{pX}, k_{d}_{_{—}}_{pY}, k_{d}_{_{—}}_{pZ}) | 8.4 | days⁻¹ |
| Gene concentration (gX, gY, gZ) | 10 | pmol |
| Probability that activator is bound (β_{X}, β_{Y}, β_{Z}) | 1 | |
| Hill coefficients (Kx, Ky, Kz) | 1 | |
| Rate of siRNA degradation (k_{d}_{_{—}}ₛᵢᵣₙₐ₁, k_{d}_{_{—}}ₛᵢᵣₙₐ₂) | 0.34 | day⁻¹ |
| Rate of target mRNA degradation by siRNA | 16.6 | days⁻¹ |
| (k_{d}_{_{—}}ₛᵢᵣₙₐ₁_{_{—}}ₘᵣₙₐ, k_{d}_{_{—}}ₛᵢᵣₙₐ₂_{_{—}}ₘᵣₙₐ) | | |
| Initial concentration of siRNA on nanoantennas | 1 | pmol |

What is claimed is:

1. A method for reconfiguring gene circuits in a cell, comprising:
introducing plasmon resonant nanoparticles functionalized with a cationic lipid coating and adsorbed biomolecules into cells; and
exposing the cells to light with a wavelength matching a plasmon resonance wavelength of the nanoparticles to release the adsorbed biomolecules from the functionalized nanoparticles into the cells;
wherein the released biomolecule influences genetic circuits of the cell.

2. The method as recited in claim 1, further comprising:
introducing a second plasmon resonant nanoparticle functionalized with a cationic lipid coating and adsorbed biomolecules of a second biomolecule into cells; and
exposing the cells to light with a wavelength matching a plasmon resonance wavelength of the second nanoparticles to release the adsorbed second biomolecules from the second functionalized nanoparticles into the cells;
wherein said second nanoparticle has a plasmon resonance wavelength that is different than any other nanoparticle plasmon resonance wavelength of nanoparticles introduced into the cell.

3. The method as recited in claim 2, further comprising:
introducing a third plasmon resonant nanoparticle functionalized with a cationic lipid coating and adsorbed biomolecules of a third biomolecule into cells; and
exposing the cells to light with a wavelength matching a plasmon resonance wavelength of the third nanoparticles to release the adsorbed third biomolecules from the third functionalized nanoparticles into the cells;
wherein said third nanoparticle has a plasmon resonance wavelength that is different than any other nanoparticle plasmon resonance wavelength of nanoparticles introduced into the cell.

4. A method as recited in claim 1, wherein said biomolecule is selected from the group of biomolecules consisting essentially of transcription factor proteins, RNA, DNA and oligonucleotides.

5. A method as recited in claim 1, wherein said biomolecule is a small interfering RNA (siRNA) biomolecule.

6. A method as recited in claim 1, wherein said lipid coating comprises a cationic phospholipid bilayer coating.

7. A method as recited in claim 1, wherein said nanoparticle is a nanorod with an aspect ratio between 2.0 and 8.0.

8. A method as recited in claim 1, wherein said nanoparticle is a nanorod with a plasmon resonance wavelength in the near infrared (NIR) spectrum.

9. A method as recited in claim 3, wherein a first, second or third biomolecule are the same biomolecule.

10. A method as recited in claim 1, further comprising:
introducing said nanoparticles simultaneously into the cell; and
temporally controlling the light exposure and release of said biomolecules from said nanoparticles in the cell.

11. A method as recited in claim 1, further comprising:
acquiring one or more groups of small interfering RNA (siRNA) biomolecules, each siRNA group configured to interfere with transcription of a different gene in a cell; and
adhering the different groups of siRNA biomolecules onto coated nanoparticles that have a single plasmon resonant wavelength;
wherein groups of different siRNA biomolecules are released with a single light exposure.

12. A method for reconfiguring gene circuits in a cell, comprising:
providing groups of plasmon resonant nanoparticles, each group of nanoparticles having a discrete plasmon resonance wavelength;
coating the groups of nanoparticles with a cationic lipid coating;
reversibly coupling different biomolecules with each group of coated nanoparticles to form groups of addressable biomolecular nanoantennas;
depositing the biomolecular nanoantennas into cells; and
exposing the cells to light with a wavelength matching a plasmon resonance wavelength of each group of nanoparticles to release the coupled biomolecules from the nanoantennas;
wherein the released biomolecule influences genetic circuits of the cell; and
wherein the release of each group of biomolecules is temporally controlled by the timing of light exposure.

13. A method as recited in claim 12, wherein said lipid coating comprises a cationic phospholipid bilayer coating.

14. A method as recited in claim 12, wherein said nanoparticles are nanorods with an aspect ratio of between 2.0 and 8.0.

15. A method as recited in claim 12, wherein said nanoparticle is a nanorod with a plasmon resonance wavelength in the near infrared (NIR) spectrum.

16. A method as recited in claim 12, wherein said biomolecules are selected from the group of biomolecules consisting essentially of transcription factor proteins, RNA, DNA and oligonucleotides.

17. A method as recited in claim 12, further comprising:
selecting a gene circuit for manipulation;
selecting biomolecules that will initiate or terminate the transcription of genes in the selected gene circuit; and
providing the selected biomolecules for adsorption with coated nanoparticles.

18. A method as recited in claim 17, further comprising:
selecting biomolecules that will increase or decrease the rate of transcription of genes in the selected gene circuit.

19. A biomolecule carrier for introducing biomolecules into a cell, comprising:
a plasmonic nanoparticle with a plasmon resonant wavelength; and
a cationic lipid coating on the exterior surfaces of the nanoparticle.

20. A biomolecule carrier as recited in claim 19, wherein said lipid coating comprises a cationic phospholipid bilayer coating.

21. A biomolecule carrier as recited in claim 19, wherein said nanoparticles are nanorods with an aspect ratio of between 2.0 and 8.0.

22. A biomolecule carrier as recited in claim 19, wherein said nanoparticle is a nanorod with a plasmon resonance wavelength in the near infrared (NIR) spectrum.
